(19) United States
(12) Patent Application Publication
(10) Pub. No.: US 2024/0212335 A1
(43) Pub. Date: Jun. 27, 2024

(54) METHOD AND SYSTEM FOR IMAGE PROCESSING BASED ON CONVOLUTIONAL NEURAL NETWORK

(71) Applicant: Exo Imaging, Inc., Santa Clara, CA (US)

(72) Inventors: Xuebin QIN, I, Edmonton (CA); Masood DEHGHAN, Edmonton (CA); Dornoosh ZONOOBI, Edmonton (CA)

(21) Appl. No.: 18/557,233

(22) PCT Filed: Oct. 14, 2021
(86) PCT No.: PCT/SG2021/050623

§ 371 Date: Oct. 25, 2023

(51) Int. Cl. G06V 10/82; G06V 10/42

(52) U.S. Cl. CPC G06V 10/82; G06V 10/42

(57) ABSTRACT

There is provided a method of image processing based on a convolutional neural network (CNN). The method includes: receiving an input image; performing a plurality of feature extraction operations using a plurality of convolution layers, respectively, of the CNN based on the input image to produce a plurality of output feature maps, respectively; and producing an output image for the input image based on the plurality of output feature maps of the plurality of convolution layers. In particular, for each of the plurality of feature extraction operations, performing the feature extraction operation using the convolution layer includes: producing the output feature map of the convolution layer based on an input feature map received by the convolution layer and a plurality of weighted coordinate maps; producing the plurality of weighted coordinate maps based on a plurality of coordinate maps and a spatial attention map; and producing the spatial attention map based on the input feature map received by the convolution layer for modifying coordinate information in each of the plurality of coordinate maps to produce the plurality of weighted coordinate maps. There is also provided a corresponding system for image processing based on a CNN.

### TECHNICAL FIELD

The present invention generally relates to a method and a system for image processing based on a convolutional neural network (CNN).

### BACKGROUND

Convolutional neural network (CNN) is a class of artificial neural networks that is well known in the art and has been applied in a variety of domains for prediction purposes, and in particular, in image processing for various prediction applications, such as image segmentation and image classification. Although CNN may generally be understood to be applicable in a variety of domains for various prediction applications, the use of CNN in various prediction applications may not always provide satisfactory prediction results (e.g., not sufficiently accurate in image segmentation or image classification) and it may be difficult or challenging to obtain satisfactory prediction results.

As an example, medical ultrasound imaging is a safe and non-invasive real-time imaging modality that provides images of structures of the human body using high-frequency sound waves. Compared to other imaging modalities, such as Computed Tomography (CT) and Magnetic Resonance Imaging (MRI), ultrasound imaging is relatively cheap, portable and more prevalent, and hence it is widely considered to become the stethoscope of the 21^{st} century. However, ultrasound images may be obtained from a handheld probe and thus are operator-dependant and susceptible to a large number of artifacts, such as heavy speckle noise, shadowing and blurred boundaries. This increases the difficulties in the segmentation of tissue structures (e.g., anatomical structures) of interest from neighboring tissues. A number of conventional methods (e.g., active contours, graph cut, super-pixel and deep models (e.g., fully convolutional network (FCN), U-Net, and so on) have been proposed and adapted for ultrasound image segmentation. However, due to the noisiness nature of ultrasound images, such conventional methods usually produce inferior results. Although deep models have achieved great improvements against traditional methods, accurate segmentation of soft-tissue structures from ultrasound images remains a challenging task.

Another problem associated with the segmentation of ultrasound images using single deep models is that they generally produce results with high biases due to blurred boundaries and textures, and high variances due to noise and inhomogeneity. To reduce both biases and variances, multi-model ensemble approaches, such as bagging, boosting, and so on, have been proposed. However, training multiple models for ensembling is computationally expensive. To address this, it had previously been proposed to train a model with one pass while saving multiple sets of model weights along the optimization path by learning rates annealing. However, such a method still requires running the inference process multiple times. In an attempt to address this issue, a number of multi-stage predict-refine deep models (e.g., HourglassNet, CU-Net, R³-Net, BASNet) have been developed to predict and gradually refine the segmentation results through their cascaded modules. Although such a strategy may be able to reduce the segmentation biases, it has a limited impact on the variances, which means that their average performance on the whole dataset may appear good but they are less likely to produce stable prediction for different input images.

A need therefore exists to provide a method and a system for image processing based on a CNN, that seek to overcome, or at least ameliorate, one or more problems associated with conventional methods and systems for image processing based on a CNN, and in particular, enhancing or improving the predictive capability (e.g., accuracy of prediction results) associated with image processing based on a CNN, such as but not limited to, image segmentation. It is against this background that the present invention has been developed.

### SUMMARY

According to a first aspect of the present invention, there is provided a method of image processing based on a CNN, using at least one processor, the method comprising:
receiving an input image;
performing a plurality of feature extraction operations using a plurality of convolution layers, respectively, of the CNN based on the input image to produce a plurality of output feature maps, respectively; and
producing an output image for the input image based on the plurality of output feature maps of the plurality of convolution layers,
wherein for each of the plurality of feature extraction operations, performing the feature extraction operation using the convolution layer comprises:
   producing the output feature map of the convolution layer based on an input feature map received by the convolution layer and a plurality of weighted coordinate maps;
   producing the plurality of weighted coordinate maps based on a plurality of coordinate maps and a spatial attention map; and
   producing the spatial attention map based on the input feature map received by the convolution layer for modifying coordinate information in each of the plurality of coordinate maps to produce the plurality of weighted coordinate maps.

According to a second aspect of the present invention, there is provided a system for image processing based on a CNN, the system comprising: a memory; and at least one processor communicatively coupled to the memory and configured to perform the method of image processing based on a CNN according to the above-mentioned first aspect of the present invention.

According to a third aspect of the present invention, there is provided a computer program product, embodied in one or more non-transitory computer-readable storage mediums, comprising instructions executable by at least one processor to perform the method of image processing based on a CNN according to the above-mentioned first aspect of the present invention.

According to a fourth aspect of the present invention, there is provided a method of segmenting a tissue structure in an ultrasound image using a CNN, using at least one processor, the method comprising:
performing the method of image processing based on a CNN according to the above-mentioned first aspect of the present invention, wherein
the input image is the ultrasound image including the tissue structure; and
the output image has the tissue structure segmented and is a result of an inference on the input image using the CNN.

According to a fifth aspect of the present invention, there is provided a system for image processing based on a CNN, the system comprising: a memory; and at least one processor communicatively coupled to the memory and configured to perform the method of segmenting a tissue structure in an ultrasound image using a CNN according to the above-mentioned fourth aspect of the present invention.

According to a sixth aspect of the present invention, there is provided a computer program product, embodied in one or more non-transitory computer-readable storage mediums, comprising instructions executable by at least one processor to perform the method of segmenting a tissue structure in an ultrasound image using a CNN according to the above-mentioned fourth aspect of the present invention.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1 depicts a schematic flow diagram of a method of image processing based on a CNN, according to various embodiments of the present invention;

FIG. 2 depicts a schematic block diagram of a system for image processing based on a CNN, according to various embodiments of the present invention;

FIG. 3 depicts a schematic block diagram of an exemplary computer system which may be used to realize or implement the system for image processing based on a CNN, according to various embodiments of the present invention;

FIGS. 4A and 4B depict an example network architecture of an example CNN, according to various example embodiments of the present invention;

FIG. 5 shows a table (Table 1) illustrating example detailed configurations of the prediction module and the refinement module of the example CNN, according to various example embodiments of the present invention;

FIG. 6 depicts a schematic block diagram of a residual U-block (RSU), according to various example embodiments of the present invention;

FIGS. 7A and 7B depict schematic block diagrams of a residual block (FIG. 7A) and the RSU (FIG. 7B) according to various example embodiments;

FIGS. 8A and 8B depict schematic block diagrams of an original coordinate convolution (CoordConv) (FIG. 8A) and the attentive coordinate convolution (AC-Conv) (FIG. 8B) according to various example embodiments of the present invention;

FIGS. 9A and 9B depict schematic block diagrams of a conventional cascaded refinement module and the parallel refinement module according to various example embodiments of the present invention;

FIG. 10 depicts a schematic drawing of a thyroid gland and an ultrasound scanning protocol, along with corresponding ultrasound images with manually labelled thyroid lobe overlay, according to various example embodiments of the present invention;

FIG. 11 depicts a table (Table 2) illustrating the number of volumes and the corresponding slices (images) in each subset of ultrasound images, according to various example embodiments of the present invention;

FIG. 12 depicts a table (Table 3) showing the quantitative evaluation or comparison of the example CNN according to various example embodiments of the present invention with other state-of-the-art segmentation models on transverse (TRX) and sagittal (SAG) test sets;

FIGS. 13A to 13L show the sample segmentation results on TRX thyroid images using the example CNN, according to various example embodiments of the present invention;

FIGS. 14A to 14L show the sample segmentation results on SAG thyroid images using the example CNN, according to various example embodiments of the present invention;

FIGS. 15A and 15B show plots of the success rate curves of the example CNN according to various example embodiments of the present invention and other state-of-the-art models on TRX images and SAG images, respectively; and

FIG. 16 depicts a table (Table 4) showing the ablation studies conducted on different convolution blocks and refinement architectures.

### DETAILED DESCRIPTION

Various embodiments of the present invention provide a method and a system for image processing based on a convolutional neural network (CNN), and more particularly, a deep CNN. CNN is a class or type of artificial neural networks, which may also be referred to as a CNN model, or simply as a model. For example, as described in the background, although CNN may generally be understood to be applicable in a variety of domains for various prediction applications, the use of CNN in various prediction applications may not always provide satisfactory prediction results (e.g., not sufficiently accurate in image segmentation or image classification) and it may be difficult or challenging to obtain satisfactory prediction results. As an example, an ultrasound image, including a tissue structure (e.g., an anatomical structure or other types of tissue structure, such as tumour), is noisy and conventional methods for segmenting such an ultrasound image based on a CNN have been found to produce inferior results. Accordingly, various embodiments of the present invention provide a method and a system for image processing based on a CNN, that seek to overcome, or at least ameliorate, one or more problems associated with conventional methods and systems for image processing based on a CNN, and in particular, enhancing or improving the predictive capability (e.g., accuracy of prediction results) associated with image processing based on a CNN, such as but not limited to, image segmentation.

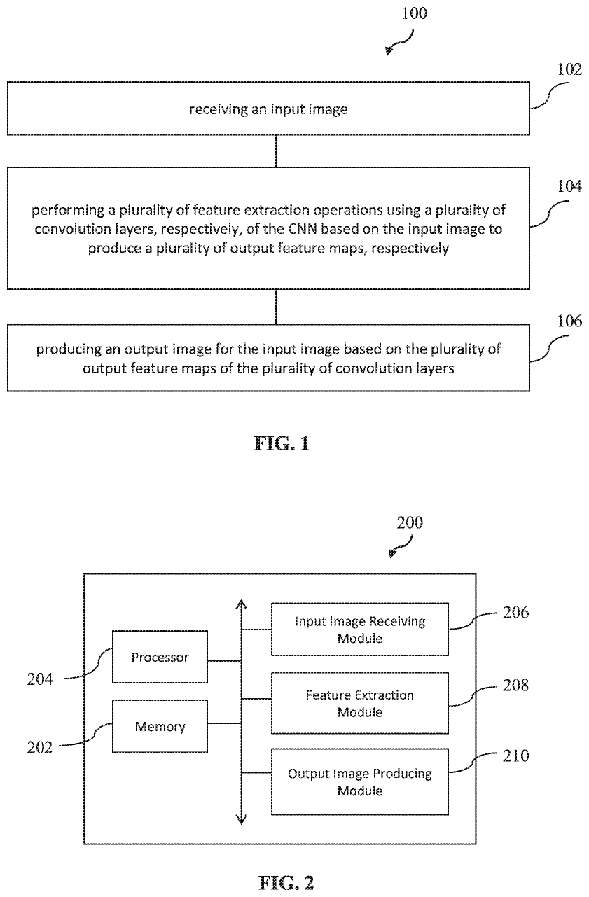

FIG. 1 depicts a schematic flow diagram of a method 100 of image processing based on a CNN, using at least one processor, according to various embodiments of the present invention. The method 100 comprises: receiving (at 102) an input image; performing (at 104) a plurality of feature extraction operations using a plurality of convolution layers, respectively, of the CNN based on the input image to produce a plurality of output feature maps, respectively; and producing (at 106) an output image for the input image based on the plurality of output feature maps of the plurality of convolution layers. In particular, for each of the plurality of feature extraction operations, performing the feature extraction operation using the convolution layer comprises: producing the output feature map of the convolution layer based on an input feature map received by the convolution layer and a plurality of weighted coordinate maps; producing the plurality of weighted coordinate maps based on a plurality of coordinate maps and a spatial attention map; and producing the spatial attention map based on the input feature map received by the convolution layer for modifying coordinate information in each of the plurality of coordinate maps to produce the plurality of weighted coordinate maps.

Accordingly, the method 100 of image processing has advantageously been found to enhance or improve predictive capability, especially in relation to image segmentation, and more particularly, in relation to ultrasound image segmentation. In particular, by performing the feature extraction operation using the corresponding convolution layer in the manner as described above, not only does the associated convolution operation have access to coordinate information (through the use of coordinate maps (extra coordinate channels)), the associated convolution operation is able to focus more (i.e., added attention) on certain coordinates that may be beneficial for the feature extraction operation (through the use of the spatial attention map, which may also be referred to simply as an attention map), whereby such added focus (i.e., added attention) is guided by the input feature map received by the convolution layer through the spatial attention map derived from the input feature map. Accordingly, not only does the associated convolution operation know where it is spatially (e.g., in the Cartesian space), the associated convolution operation knows where to focus more through the spatial attention map. For example, through the spatial attention map, extra weights may be added to certain coordinates that may require more focus or attention, and weights may be reduced to certain coordinates that may require less focus or attention, as guided by the input feature map (e.g., more important portions of the input feature map may thus receive more attention in the feature extraction operation), thereby resulting in the associated convolution operation of the convolution layer advantageously having attentive coordinate guidance. Accordingly, such a feature extraction operation using such a convolution layer having attentive coordinate guidance may be referred to as an attentive coordinate-guided convolution (AC-Conv) and such a convolution layer having attentive coordinate guidance may be referred to as an AC-Conv layer. In this regard, with attentive coordinate guidance, the method 100 of image processing has advantageously been found to enhance or improve predictive capability. These advantages or technical effects, and/or other advantages or technical effects, will become more apparent to a person skilled in the art as the method 100 of image processing, as well as the corresponding system for image processing, is described in more detail according to various embodiments and example embodiments of the present invention.

In various embodiments, the above-mentioned producing the spatial attention map comprises: performing a first convolution operation based on the input feature map received by the convolution layer to produce a convolved feature map; and applying an activation function based on the convolved feature map to produce the spatial attention map.

In various embodiments, the activation function is a sigmoid activation function.

In various embodiments, the above-mentioned producing the plurality of weighted coordinate maps comprises multiplying each of the plurality of coordinate maps with the spatial attention map so as to modify the coordinate information in each of the plurality of coordinate maps.

In various embodiments, the plurality of coordinate maps comprises a first coordinate map comprising coordinate information with respect to a first dimension and a second coordinate map comprising coordinate information with respect to a second dimension, the first and second dimensions being two dimensions over which the first convolution operation is configured to perform.

In various embodiments, the above-mentioned producing the output feature map of the convolution layer comprises: concatenating the input feature map received by the convolution layer and the plurality of weighted coordinate maps channel-wise to form a concatenated feature map; and performing a second convolution operation based on the concatenated feature map to produce the output feature map of the convolution layer.

In various embodiments, the CNN comprises a prediction sub-network comprising at least one convolution layer of the plurality of convolution layers of the CNN. In this regard, the method 100 further comprises producing a set of predicted feature maps using the prediction sub-network based on the input image, the above-mentioned producing the set of predicted feature maps comprising performing at least one feature extraction operation of the plurality of feature extraction operations using the at least one convolution layer of the prediction sub-network. Furthermore, a plurality of predicted feature maps of the set of predicted feature maps have different spatial resolution levels.

In various embodiments, the prediction sub-network has an encoder-decoder structure comprising a set of encoder blocks and a set of decoder blocks. The set of encoder blocks of the prediction sub-network comprises a plurality of encoder blocks and the set of decoder blocks of the prediction sub-network comprises a plurality of decoder blocks. In this regard, the method 100 further comprises: producing, for each of the plurality of encoder blocks of the prediction sub-network, a downsampled feature map using the encoder block based on an input feature map received by the encoder block; and producing, for each of the plurality of decoder blocks of the prediction sub-network, an upsampled feature map using the decoder block based on an input feature map and the downsampled feature map produced by the encoder block corresponding to the decoder block received by the decoder block.

In various embodiments, the above-mentioned producing the set of predicted feature maps using the prediction sub-network comprises producing the plurality of predicted feature maps based on the plurality of upsampled feature maps produced by the plurality of decoder blocks, respectively.

In various embodiments, the above-mentioned producing the downsampled feature map using the encoder block of the prediction sub-network comprises: extracting multi-scale features based on the input feature map received by the encoder block; and producing the downsampled feature map based on the extracted multi-scale features extracted by the encoder block. In various embodiments, the above-mentioned producing the upsampled feature map using the decoder block of the prediction sub-network comprises: extracting multi-scale features based on the input feature map and the downsampled feature map produced by the encoder block corresponding to the decoder block received by the decoder block; and producing the upsampled feature map based on the extracted multi-scale features extracted by the decoder block.

In various embodiments, each of the plurality of encoder blocks of the prediction sub-network comprises at least one convolution layer of the plurality of convolution layers of the CNN, and the above-mentioned producing the downsampled feature map using the encoder block of the prediction sub-network comprises performing at least one feature extraction operation of the plurality of feature extraction operations using the at least one convolution layer of the encoder block. In various embodiments, each of the plurality of decoder blocks of the prediction sub-network comprises at least one convolution layer of the plurality of convolution layers of the CNN, and the above-mentioned producing the upsampled feature map using the decoder block of the prediction sub-network comprises performing at least one feature extraction operation of the plurality of feature extraction operations using the at least one convolution layer of the decoder block.

In various embodiments, each convolution layer of each of the plurality of encoder blocks of the prediction sub-network is one of the plurality of convolution layers of the CNN. In various embodiments, each convolution layer of each of the plurality of decoder blocks of the prediction sub-network is one of the plurality of convolution layers of the CNN.

In various embodiments, each of the plurality of encoder blocks of the prediction sub-network is configured as a residual block. In various embodiments, each of the plurality of decoder blocks of the prediction sub-network is configured as a residual block.

In various embodiments, the CNN further comprises a refinement sub-network comprising at least one convolution layer of the plurality of convolution layers of the CNN. In this regard, the method 100 further comprises producing a set of refined feature maps using the refinement sub-network based on a fused feature map, the above-mentioned producing the set of refined feature maps comprising performing at least one feature extraction operation of the plurality of feature extraction operations using the at least one convolution layer of refinement sub-network. Furthermore, a plurality of refined feature maps of the set of refined feature maps have different spatial resolution levels.

In various embodiments, the method 100 further comprises concatenating the set of predicted feature maps to produce the fused feature map.

In various embodiments, the refinement sub-network comprises a plurality of refinement blocks configured to produce the plurality of refined feature maps, respectively, each of the plurality of refinement blocks having an encoder-decoder structure comprising a set of encoder blocks and a set of decoder blocks. The set of encoder blocks of the refinement sub-network comprises a plurality of encoder blocks and the set of decoder blocks of the refinement sub-network comprises a plurality of decoder blocks. In this regard, the method 100 further comprises, for each of the plurality of refinement blocks: producing, for each of the plurality of encoder blocks of the refinement block, a downsampled feature map using the encoder block based on an input feature map received by the encoder block; and producing, for each of the plurality of decoder blocks of the refinement block, an upsampled feature map using the decoder block based on an input feature map and the downsampled feature map produced by the encoder block corresponding to the decoder block received by the decoder block.

In various embodiments, the plurality of encoder-decoder structures of the plurality of refinement blocks have different heights.

In various embodiments, the above-mentioned producing the set of refined feature maps using the refinement sub-network comprises producing, for each of the plurality of refinement blocks, the refined feature map of the refinement block based on the fused feature map received by the refinement block and the upsampled feature map produced by a first decoder block of the plurality of decoder blocks of the refinement block.

In various embodiments, the above-mentioned producing the downsampled feature map using the encoder block of the refinement block comprises: extracting multi-scale features based on the input feature map received by the encoder block; and producing the downsampled feature map based on the extracted multi-scale features extracted by the encoder block. In various embodiments, the above-mentioned producing the upsampled feature map using the decoder block of the refinement block comprises: extracting multi-scale features based on the input feature map and the downsampled feature map produced by the encoder block of the refinement block corresponding to the decoder block received by the decoder block; and producing the upsampled feature map based on the extracted multi-scale features extracted by the decoder block.

In various embodiments, for each of the plurality of refinement blocks: each of the plurality of encoder blocks of the refinement block comprises at least one convolution layer of the plurality of convolution layers of the CNN, and the above-mentioned producing the downsampled feature map using the encoder block of the refinement block comprises performing at least one feature extraction operation of the plurality of feature extraction operations using the at least one convolution layer of the encoder block. In various embodiments, for each of the plurality of refinement blocks: each of the plurality of decoder blocks of the refinement block comprises at least one convolution layer of the plurality of convolution layers of the CNN, and the above-mentioned producing the upsampled feature map using the decoder block of the refinement block comprises performing at least one feature extraction operation of the plurality of feature extraction operations using the at least one convolution layer of the decoder block.

In various embodiments, each convolution layer of each of the plurality of encoder blocks of the refinement block is one of the plurality of convolution layers of the CNN. In various embodiments, each convolution layer of each of the plurality of decoder blocks of the refinement block is one of the plurality of convolution layers of the CNN.

In various embodiments, for each of the plurality of refinement blocks, each of the plurality of encoder blocks of the refinement block is configured as a residual block, and each of the plurality of decoder blocks of the refinement block is configured as a residual block.

In various embodiments, the output image is produced based on the set of refined feature maps.

In various embodiments, the output image is produced based on an average of the set of refined feature maps.

In various embodiments, the above-mentioned receiving (at 102) the input image comprises receiving a plurality of input images, each of the plurality of input images being a labeled image so as to train the CNN to obtain a trained CNN. In this regard, for each of the plurality of input images: performing the plurality of feature extraction operations using the plurality of convolution layers, respectively, of the CNN based on the input image to produce the plurality of output feature maps, respectively; and producing the output image for the input image based on the plurality of output feature maps of the plurality of convolution layers.

In various embodiments, the label image is a labeled ultrasound image including a tissue structure.

In various embodiments, the output image is a result of an inference on the input image using the CNN.

In various embodiments, the input image is an ultrasound image including a tissue structure.

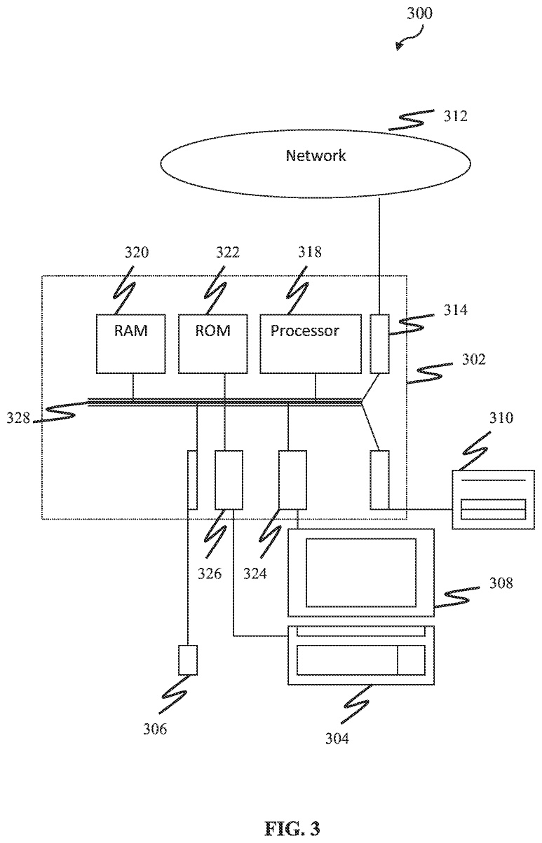

FIG. 2 depicts a schematic block diagram of a system 200 for image processing based on a CNN, according to various embodiments of the present invention, corresponding to the method 100 of image processing as described hereinbefore with reference to FIG. 1 according to various embodiments of the present invention. The system 200 comprises: a memory 202; and at least one processor 204 communicatively coupled to the memory 202 and configured to perform the method 100 of image processing as described herein according to various embodiments of the present invention. Accordingly, in various embodiments, the at least one processor 204 is configured to: receive an input image; perform a plurality of feature extraction operations using a plurality of convolution layers, respectively, of the CNN based on the input image to produce a plurality of output feature maps, respectively; and produce an output image for the input image based on the plurality of output feature maps of the plurality of convolution layers. In particular, as described hereinbefore, for each of the plurality of feature extraction operations, performing the feature extraction operation using the convolution layer comprises: producing the output feature map of the convolution layer based on an input feature map received by the convolution layer and a plurality of weighted coordinate maps; producing the plurality of weighted coordinate maps based on a plurality of coordinate maps and a spatial attention map; and producing the spatial attention map based on the input feature map received by the convolution layer for modifying coordinate information in each of the plurality of coordinate maps to produce the plurality of weighted coordinate maps.

It will be appreciated by a person skilled in the art that the at least one processor 204 may be configured to perform various functions or operations through set(s) of instructions (e.g., software modules) executable by the at least one processor 204 to perform various functions or operations. Accordingly, as shown in FIG. 2, the system 200 may comprise an input image receiving module (or an input image receiving circuit) 206 configured to receive an input image; a feature extraction module (or a feature extraction circuit) 208 configured to perform a plurality of feature extraction operations using a plurality of convolution layers, respectively, of the CNN based on the input image to produce a plurality of output feature maps, respectively; and an output image producing module (or an output image producing circuit) 210 configured to produce an output image for the input image based on the plurality of output feature maps of the plurality of convolution layers.

It will be appreciated by a person skilled in the art that the above-mentioned modules are not necessarily separate modules, and one or more modules may be realized by or implemented as one functional module (e.g., a circuit or a software program) as desired or as appropriate without deviating from the scope of the present invention. For example, two or more of the input image receiving module 206, the feature extraction module 208 and the output image producing module 210 may be realized (e.g., compiled together) as one executable software program (e.g., software application or simply referred to as an “app”), which for example may be stored in the memory 202 and executable by the at least one processor 204 to perform various functions/operations as described herein according to various embodiments of the present invention.

In various embodiments, the system 200 for image processing corresponds to the method 100 of image processing as described hereinbefore with reference to FIG. 1 according to various embodiments, therefore, various functions or operations configured to be performed by the least one processor 204 may correspond to various steps or operations of the method 100 of image processing as described hereinbefore according to various embodiments, and thus need not be repeated with respect to the system 200 for image processing for clarity and conciseness. In other words, various embodiments described herein in context of the methods are analogously valid for the corresponding systems, and vice versa.

For example, in various embodiments, the memory 202 may have stored therein the input image receiving module 206, the feature extraction module 208 and/or the output image producing module 210, which respectively correspond to various steps (or operations or functions) of the method 100 of image processing as described herein according to various embodiments, which are executable by the at least one processor 204 to perform the corresponding functions or operations as described herein.

In various embodiments, there is provided a method of segmenting a tissue structure in an ultrasound image using a CNN, using at least one processor, according to various embodiments of the present invention. The method comprises: performing the method 100 of image processing based on a CNN as described hereinbefore according to various embodiments, whereby the input image is the ultrasound image including the tissue structure; and the output image has the tissue structure segmented and is a result of an inference on the input image using the CNN.

In various embodiments, the CNN is trained as described hereinbefore according to various embodiments. That is, the CNN is the above-mentioned trained CNN.

In various embodiments, there is provided a system for segmenting a tissue structure in an ultrasound image using a CNN, according to various embodiments, corresponding to the above-mentioned method of segmenting a tissue structure in an ultrasound image according to various embodiments, The system comprises: a memory; and at least one processor communicatively coupled to the memory and configured to perform the above-mentioned method of segmenting a tissue structure in an ultrasound image. In various embodiments, the system for segmenting a tissue structure in an ultrasound image may be the same as the system 200 for image processing, whereby the input image is the ultrasound image including the tissue structure; and the output image has the tissue structure segmented and is a result of an inference on the input image using the CNN.

A computing system, a controller, a microcontroller or any other system providing a processing capability may be provided according to various embodiments in the present disclosure. Such a system may be taken to include one or more processors and one or more computer-readable storage mediums. For example, the system 200 for image processing described hereinbefore may include a processor (or controller) 204 and a computer-readable storage medium (or memory) 202 which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of the respective functions may also be understood as a “circuit” in accordance with various embodiments. Similarly, a “module” may be a portion of a system according to various embodiments and may encompass a “circuit” as described above, or may be understood to be any kind of a logic-implementing entity.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, description or discussions utilizing terms such as “receiving”, “performing”, “producing”, “multiplying”, “concatenating”, “extracting” or the like, refer to the actions and processes of a computer system, or similar electronic device, that manipulates and transforms data represented as physical quantities within the computer system into other data similarly represented as physical quantities within the computer system or other information storage, transmission or display devices.

The present specification also discloses a system (e.g., which may also be embodied as a device or an apparatus), such as the system 200 for image processing, for performing various operations/functions of various methods described herein. Such a system may be specially constructed for the required purposes, or may comprise a general purpose computer or other device selectively activated or reconfigured by a computer program stored in the computer. The algorithms presented herein are not inherently related to any particular computer or other apparatus. Various general-purpose machines may be used with computer programs in accordance with the teachings herein. Alternatively, the construction of more specialized apparatus to perform various method steps may be appropriate.

In addition, the present specification also at least implicitly discloses a computer program or software/functional module, in that it would be apparent to the person skilled in the art that individual steps of various methods described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the scope of the invention. It will be appreciated by a person skilled in the art that various modules described herein (e.g., the input image receiving module 206, the feature extraction module 208 and/or the output image producing module 210) may be software module(s) realized by computer program(s) or set(s) of instructions executable by a computer processor to perform the required functions, or may be hardware module(s) being functional hardware unit(s) designed to perform the required functions. It will also be appreciated that a combination of hardware and software modules may be implemented.

Furthermore, one or more of the steps of a computer program/module or method described herein may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer program when loaded and executed on such a general-purpose computer effectively results in an apparatus that implements the steps of the methods described herein.

In various embodiments, there is provided a computer program product, embodied in one or more computer-readable storage mediums (non-transitory computer-readable storage medium(s)), comprising instructions (e.g., the input image receiving module 206, the feature extraction module 208 and/or the output image producing module 210) executable by one or more computer processors to perform the method 100 of image processing, as described herein with reference to FIG. 1 according to various embodiments. Accordingly, various computer programs or modules described herein may be stored in a computer program product receivable by a system therein, such as the system 200 for image processing as shown in FIG. 2, for execution by at least one processor 204 of the system 200 to perform various functions.

In various embodiments, there is provided a computer program product, embodied in one or more computer-readable storage mediums (non-transitory computer-readable storage medium(s)), comprising instructions executable by one or more computer processors to perform the above-mentioned method of segmenting a tissue structure in an ultrasound image according to various embodiments. Accordingly, various computer programs or modules described herein may be stored in a computer program product receivable by a system therein, such as the above-mentioned system for segmenting a tissue structure in an ultrasound image, for execution by at least one processor of the system to perform various functions.

Software or functional modules described herein may also be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the software or functional module(s) described herein can also be implemented as a combination of hardware and software modules.

In various embodiments, the system 200 for image processing may be realized by any computer system (e.g., desktop or portable computer system) including at least one processor and a memory, such as a computer system 300 as schematically shown in FIG. 3 as an example only and without limitation. Various methods/steps or functional modules may be implemented as software, such as a computer program being executed within the computer system 300, and instructing the computer system 300 (in particular, one or more processors therein) to conduct various functions or operations as described herein according to various embodiments. The computer system 300 may comprise a computer module 302, input modules, such as a keyboard and/or a touchscreen 304 and a mouse 306, and a plurality of output devices such as a display 308, and a printer 310. The computer module 302 may be connected to a computer network 312 via a suitable transceiver device 314, to enable access to e.g., the Internet or other network systems such as Local Area Network (LAN) or Wide Area Network (WAN). The computer module 302 in the example may include a processor 318 for executing various instructions, a Random Access Memory (RAM) 320 and a Read Only Memory (ROM) 322. The computer module 302 may also include a number of Input/Output (I/O) interfaces, for example I/O interface 324 to the display 308, and I/O interface 326 to the keyboard 304. The components of the computer module 302 typically communicate via an interconnected bus 328 and in a manner known to the person skilled in the relevant art.

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Any reference to an element or a feature herein using a designation such as “first”, “second” and so forth does not limit the quantity or order of such elements or features, unless stated or the context requires otherwise. For example, such designations may be used herein as a convenient way of distinguishing between two or more elements or instances of an element. Thus, a reference to first and second elements does not necessarily mean that only two elements can be employed, or that the first element must precede the second element. In addition, a phrase referring to “at least one of” a list of items refers to any single item therein or any combination of two or more items therein.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

In particular, for better understanding of the present invention and without limitation or loss of generality, various example embodiments of the present invention will now be described with respect to the input image being an ultrasound image and the image processing being for ultrasound image segmentation, that is, a method of image processing based on a CNN for segmenting a tissue structure in an ultrasound image. Although such a particular application (i.e., ultrasound image segmentation) may be preferred according to various example embodiments, it will be appreciated by a person skilled in the art that the present invention is not limited to such a particular application, and the method of image processing may be implemented in other types of applications as desired or as appropriate (e.g., for applications where the input image may be relatively noisy and/or the structure of interest has similar position and/or shape in the input images in general), such as but not limited to, image classification.

Ultrasound image segmentation is a challenging task due to existence of artifacts inherit to the modality, such as attenuation, shadowing, speckle noise, uneven textures and blurred boundaries. In this regard, various example embodiments provide a predict-refine attention network (which is a CNN) for segmentation of soft-tissue structures in ultrasound images, which may be referred to herein as ACU²E-Net or simply as the present CNN or model. The predict-refine attention network comprises: a prediction module or block (e.g., corresponding to the prediction sub-network as described hereinbefore according to various embodiments, and may be referred to herein as ACU²-Net), which includes attentive coordinate convolution (AC-Conv); and a multi-head residual refinement module or block (e.g., corresponding to the refinement sub-network as described hereinbefore according to various embodiments, and may be referred to herein as MH-RRM or E-Module), which includes a plurality of (e.g., three) parallel residual refinement modules or blocks (e.g., corresponding to the plurality of refinement blocks as described hereinbefore according to various embodiments). In various example embodiments, the AC-Conv is configured or designed to improve the segmentation accuracy by perceiving the shape and positional information of the target anatomy. By integrating the residual refinement and the ensemble strategies, the MH-RRM has advantageously been found to reduce both segmentation biases and variances, and avoid multi-pass training and inference commonly seen in ensemble methods. To demonstrate the effectiveness of the method of image processing based on the present CNN for segmentation of a tissue structure in an ultrasound image according to various example embodiments, a dataset of thyroid ultrasound scans was collected, and the present CNN was evaluated against state-of-the-art segmentation methods. Comparisons against state-of-the-art models demonstrate the competitive or improved performance of the present CNN on both the transverse and sagittal thyroid images. For example, ablation studies show that the AC-Conv and MH-RRM modules improve the segmentation Dice score of the baseline model from 79.62% to 80.97% and 83.92% while reducing the variance from 6.12% to 4.67% and 3.21%.

As described in the background, ultrasound images may be obtained from a handheld probe and thus are operator-dependant and susceptible to a large number of artifacts, such as heavy speckle noise, shadowing and blurred boundaries. This increases the difficulties in the segmentation of tissue structures (e.g., anatomical structures) of interest from neighboring tissues. A number of conventional methods (e.g., active contours, graph cut, super-pixel and deep models (e.g., fully convolutional network (FCN), U-Net, and so on) have been proposed and adapted for ultrasound image segmentation. However, due to the noisiness nature of ultrasound images, such conventional methods usually produce inferior results. Although deep models have achieved great improvements against the conventional methods, accurate segmentation of soft-tissue structures from ultrasound images remains a challenging task.

In relation to the ultrasound image segmentation, various example embodiments note that unlike general objects that are of different shapes and positions in natural image segmentation, tissue structures (e.g., anatomical structures) in ultrasound images have similar position and shape patterns. However, these geometric features are rarely used in the segmentation deep-models, because they are difficult to represent and encode. Accordingly, conventionally, how to make use of the specific geometric constraints of soft-tissues structures in ultrasound images remains a challenge. Another problem associated with the segmentation of ultrasound images using single deep models is that they generally produce results with high biases due to blurred boundaries and textures, and high variances due to noise and inhomogeneity.

Accordingly, to overcome these challenges, various example embodiments provide the above-mentioned attention-based predict-refine architecture (i.e., the present CNN), comprising a prediction module built upon the above-mentioned AC-Conv and a multi-head residual refinement module (MH-RRM). Such an attention-based predict-refine architecture advantageously exploits the anatomical positional and shape constraints presented in ultrasound images to reduce the biases and variances of segmentation results, while avoiding multi-pass training and inference. Accordingly, contributions of the present CNN include: (a) an AC-Conv configured to improve the segmentation accuracy by perceiving both geometric (e.g., shape and positional information) from ultrasound images; and/or (b) a predict-refine architecture with a MH-RRM, which improves the segmentation accuracy by integrating both an ensemble strategy and a predict-refine strategy together. As will be described later below, the method of image processing based on the present CNN for ultrasound image segmentation according to various example embodiments was tested on a dataset of thyroid ultrasound scans and achieved improved performance (e.g., accuracy) against conventional models.

### CNN Architecture

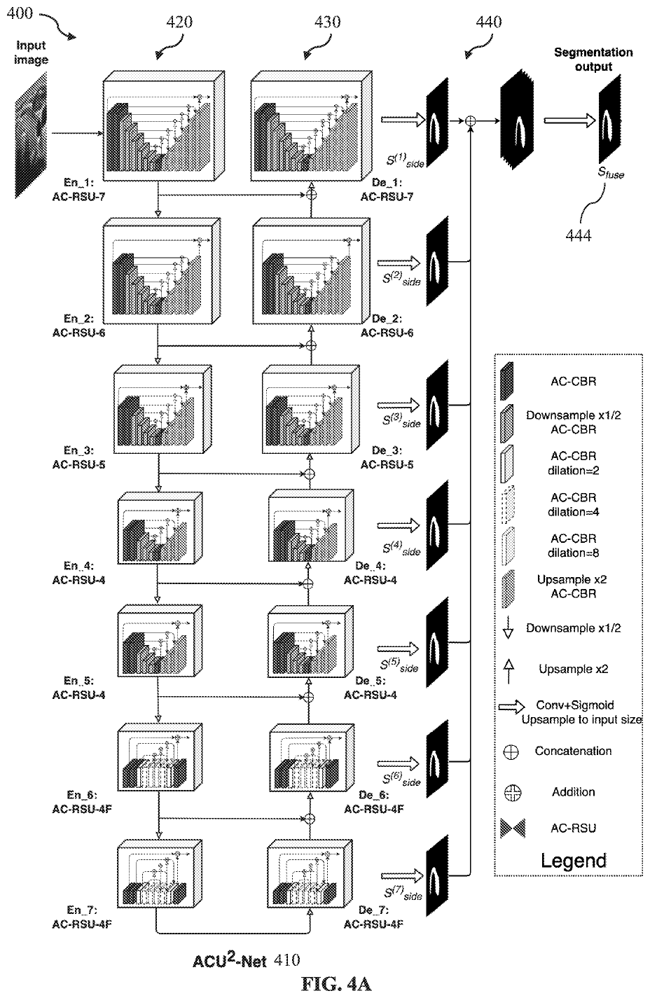

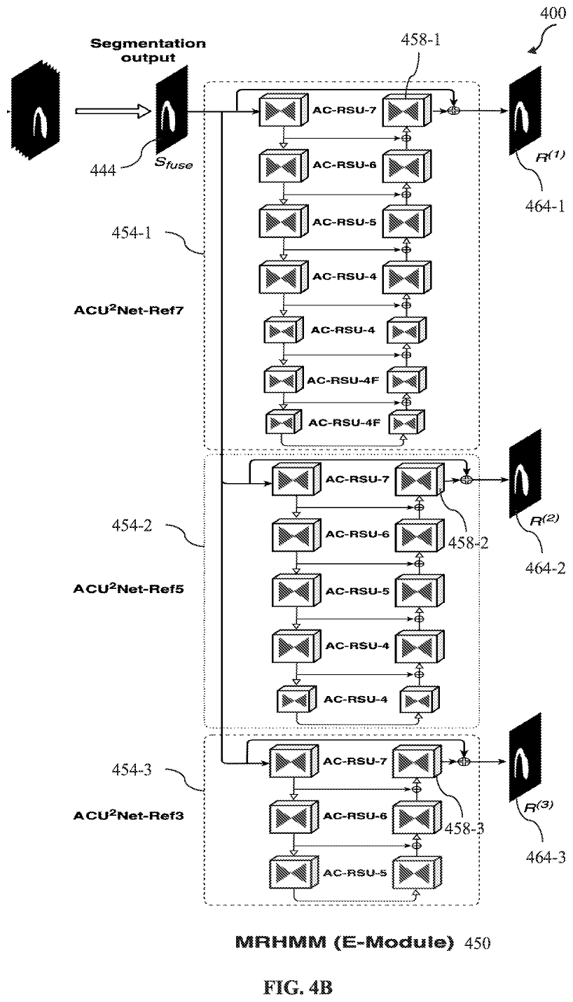

FIGS. 4A and 4B together depict an example network architecture of an example CNN 400 according to various example embodiments of the present invention. As also described hereinbefore, the example CNN 400 comprises: a prediction module or block (ACU²-Net) 410 (FIG. 4A) and a MH-RRM 450 (FIG. 4B). In various example embodiments, the prediction module 410 may be configured based on the U²-Net disclosed in Qin et al., “U²-Net: Going Deeper with nested U-structure for salient object detection, Pattern Recognition, 106: 107404, 2020 (which is herein referred to as the Qin reference, the content of which being hereby incorporated by reference in its entirety for all purposes), by replacing each plain convolution layer in the U²-Net with the AC-Conv layer described herein according to various example embodiments, so as to form an attentive coordinate-guided U²-Net (which may be referred to as ACU²-Net). In various example embodiments, the refinement module 450 comprises a set of parallel-arranged variants of the prediction module (ACU²-Net) (e.g., so as to produce refined feature maps having different spatial resolution levels). By way of an example, as shown in FIG. 4B, the refinement module 450 may be configured to have three refinement heads or blocks (being three ACU²-Net variants for producing refined feature maps having different spatial resolution levels) 454-1, 454-2, 454-3 arranged in parallel, and denoted in FIG. 4B as ACU²-Net-Ref7, ACU²-Net-Ref5 and ACU²-Net-Ref3, respectively (e.g., looks like an “E” character, and thus, such an example configuration of the refinement module 450 may also be referred to herein as an E-Module). In the legend shown in FIG. 4A, the term AC-CBR denotes AC-Conv+BacthNorm+ReLU.

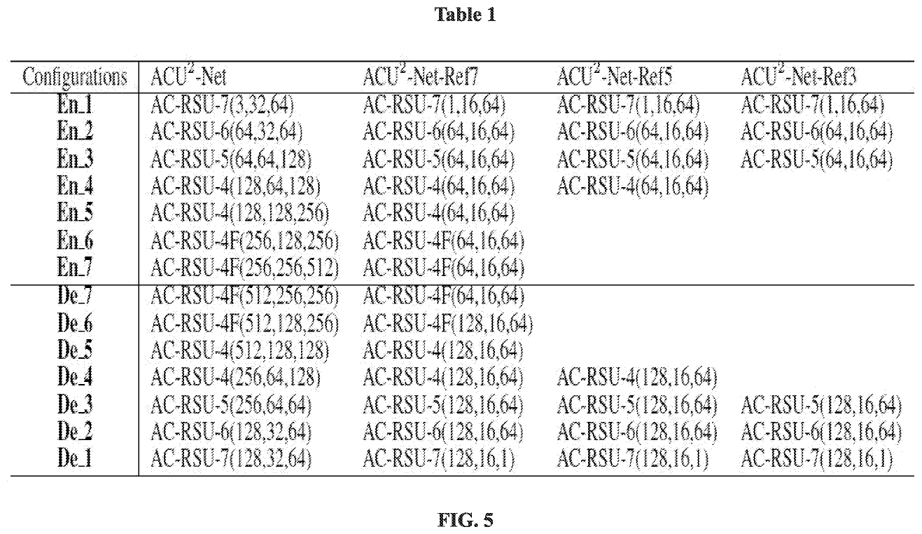

By way of an example only for illustration purpose and without limitation, FIG. 5 shows a table (Table 1) illustrating example detailed configurations of the prediction module 410 and the refinement module 450 of the example CNN 400 according to various example embodiments. The blank cells in Table 1 indicate that there are no such stages. In addition, “I”, “M” and “O” indicate the number of input channels (Cᵢₙ), middle channels and output channels (Cₒᵤₜ) of each AC-RSU block (attentive coordinate-guided residual U-block). “En_i” and “De_j” denote the encoder and decoder stages, respectively. The number “L” in “AC-RSU-L” denotes the height of the AC-RSU block. It will be appreciated by a person skilled in the art that the present invention is not limited a CNN having the example detailed configurations (or parameters) shown in FIG. 5, which are provided by way of an example only for illustration purpose and without limitations. It will be appreciated by a person skilled in the art that the parameters of the CNN can be varied or modified as desired or as appropriate for various purpose, such as but not limited to, the desired height of the encoder-decoder structure of the ACU²-Net, the desired different spatial resolution levels (and/or the desired number of different spatial resolution levels) of the predicted feature maps produced, the desired different spatial resolution levels (and/or the desired number of different spatial resolution levels) of the refined feature maps produced, the desired number of layers in the encoder or decoder block, the desired number of channels in the encoder or decoder block, and so on.

The Qin reference discloses a deep network architecture (referred to as the U²-Net) for salient object detection (SOD). The network architecture of the U²-Net is a two-level nested U-structure. The network architecture has the following advantages: (1) it is able to capture more contextual information from different scales due to the mixture of receptive fields of different sizes in the residual U-blocks (RSU blocks, which may simply be referred to as RSUs), and (2) it increases the depth of the whole architecture without significantly increasing the computational cost because of the pooling operations used in these RSU blocks. Such a network architecture enables the training of a deep network from scratch without using backbones from image classification tasks. In particular, the U²-Net is a two-level nested U-structure that is designed for SOD without using any pre-trained backbones from image classification. It can be trained from scratch to achieve competitive performance. Furthermore, the network architecture allows the network to go deeper, attain high resolution, without significantly increasing the memory and computation cost. This is achieved by a nested U-structure, whereby at the bottom level, a RSU block is configured, which is able to extract intra-stage multi-scale features without degrading the feature map resolution; and at the top level, there is a U-Net like structure (encoder-decoder structure), in which each stage is filled by a RSU block. The two-level configuration results in a nested U-structure, and an example of a nested U-structure (encoder-decoder structure) according to various example embodiments as shown in FIG. 4A, whereby as described hereinbefore, each plain convolution layer in the U²-Net is replaced by the AC-Conv layer described herein according to various example embodiments, so as to form the ACU²-Net 410.

In summary, multi-level deep feature integration methods mainly focus on developing better multi-level feature aggregation strategies. On the other hand, methods in the category of multi-scale feature extraction target at designing new modules for extracting both local and global information from features obtained by backbone networks. In this regard, the network architecture of the U²-Net or the ACU²-Net 410 is configured to directly extract multi-scale features stage by stage.

### Residual U-Block (RSU)/Attentive Coordinate-Guided Residual U-Block (AC-RSU)

Both local and global contextual information are important for salient object detection and other segmentation tasks. In modern CNN designs, such as VGG, ResNet, DenseNet and so on, small convolutional filters with size of 1×1 or 3×3 are the most frequently used components for feature extraction. They are in favor since they require less storage space and are computationally efficient. For example, the output feature maps of shallow layers only contain local features because the receptive field of 1×1 or 3×3 filters are too small to capture global information. To achieve more global information at high resolution feature maps from shallow layers, the most direct idea is to enlarge the receptive field. However, conducting multiple dilated convolutions on the input feature map (especially in the early stage) with original resolution requires too much computation and memory resources. To decrease the computation costs, the parallel configuration may be adapted from pyramid pooling modules (PPM), which uses small kernel filters on the downsampled feature maps other than the dilated convolutions on the original size feature maps. But fusion of different scale features by direct upsampling and concatenation (or addition) may lead to degradation of high resolution features.

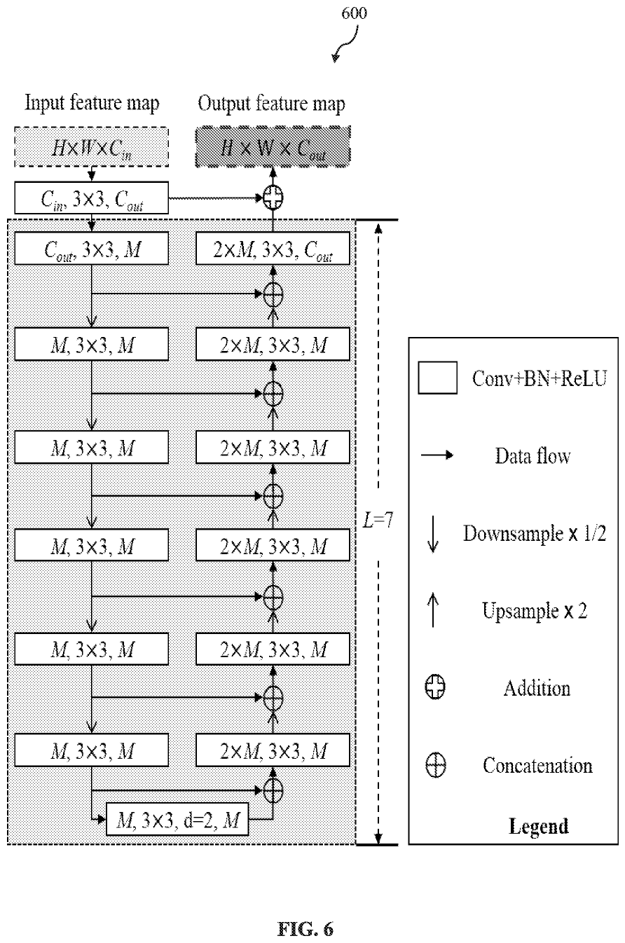

Accordingly, as described in the Qin reference, a RSU block is provided to capture intra-stage multi-scale features. By way of an example only and without limitation, an example structure of RSU-L (Cᵢₙ, M, Cₒᵤₜ) block 600 is shown in FIG. 6, where L is the number of layers in the encoder, Cᵢₙ, Cₒᵤₜ denote input and output channels, and M denotes the number of channels in the internal layers of the RSU block 600. It will be appreciated by a person skilled in the art that the RSU-L block 600 is not limited to the particular dimensions (e.g., the number of layers L) as shown in FIG. 6, which are by way of an example only and without limitation. Accordingly, the RSU block 600 comprises three components:
(i) an input convolution layer, which transforms the input feature map x (H×W×Cᵢₙ) to an intermediate map [symbol] ₁(x) with channel of Cₒᵤₜ. This is a plain convolutional layer for local feature extraction;
(ii) a U-Net like symmetric encoder-decoder structure with height of L which takes the intermediate feature map [symbol] ₁(x) as input and learns to extract and encode the multi-scale contextual information [symbol] ( [symbol] ₁(x)). [symbol] represents the U-Net like structure as shown in FIG. 6. Larger L leads to deeper residual U-block (RSU), more pooling operations, larger range of receptive fields and richer local and global features. Configuring this parameter enables extraction of multi-scale features from input feature maps with arbitrary spatial resolutions. The multi-scale features are extracted from gradually downsampled feature maps and encoded into high resolution feature maps by progressive upsampling, concatenation and convolution. This process mitigates the loss of fine details caused by direct upsampling with large scales.
(iii) a residual connection which fuses local features and the multi-scale features by the summation: [symbol] ₁(x)+ [symbol] ( [symbol] ₁(x)).

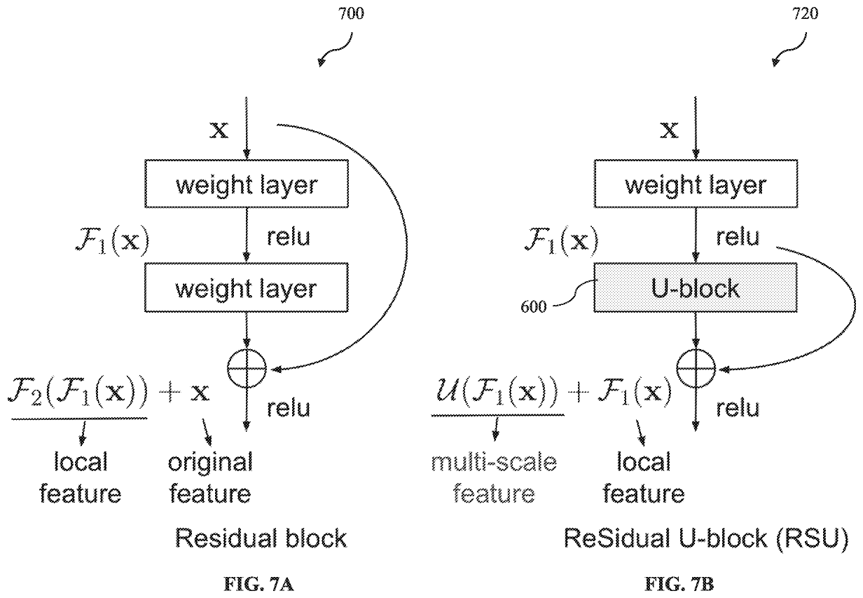

For better understanding, FIGS. 7A and 7B depict schematic drawings of an original residual block 700 (FIG. 7A) and the residual U-block (RSU) 720 (FIG. 7B) for comparison. The operation in the original residual block 700 can be summarized as [symbol] (x)= [symbol] ₂( [symbol] ₁(x))+x, where [symbol] (x) denotes the desired mapping of the input features x; [symbol] ₂, [symbol] ₁ stand for the weight layers, which are convolution operations in this setting. The main design difference between the RSU block 720 and the original residual block 700 is that the RSU block 720 replaces the plain, single-stream convolution with a U-Net like structure 600, and replace the original feature with the local feature transformed by a weight layer: [symbol] _{RSU}(x)= [symbol] ( [symbol] ₁(x))+ [symbol] ₁(x), where [symbol] represents a multi-layer U-structure 600 such as that illustrated in FIG. 6. Such a difference between the RSU block 720 and the original residual block 700 empowers the network to extract features from multiple scales directly from each RSU block. Furthermore, the computation overhead due to the U-structure is small, since most operations are applied on the downsampled feature maps.

In various example embodiments, the AC-RSU block may be formed based on (e.g., the same as or similar to) the above-described RSU block 720 (without being limited to any particular dimensions, such as the number of layers L., which may be varied or modified as desired or as appropriate, whereby each plain convolution layer in the RSU block 720 is replaced with the AC-Conv layer as described herein according to various example embodiments.

### Architecture of ACU²-Net

According to various example embodiments, there is disclosed a ACUⁿ⁻Net, whereby multiple U-Net-like structures are stacked in a nested manner. In particular, the exponential notation refers to a nested U-structure rather than cascaded stacking. Theoretically, the exponent n can be set as an arbitrary positive integer to achieve single-level or multi-level nested U-structure. But architectures with too many nested levels will be too complicated to be implemented and employed in real applications. For example, n may be set to 2 to form the ACU²-Net. The ACU²-Net has a two-level nested U-structure, and FIG. 4A depicts a schematic block diagram of an example ACU²-Net forming the prediction module 410 according to various example embodiments. The top level is a U-structure comprising a plurality of stages (the plurality of cubes in FIG. 4A), for example and without limitation, 14 stages. Each stage is filled by a configured AC-RSU block (bottom level U-structure). Accordingly, the nested U-structure enables the extraction of intra-stage multi-scale features and aggregation of inter-stage multi-level features more efficiently.

As illustrated in FIG. 4A, the prediction module (ACU²-Net) 410 has an encoder-decoder structure comprising a set of encoder blocks 420 and a set of decoder blocks 430. By way of an example only and without limitation, the prediction module 410 comprises three parts: (1) a multi-stage (e.g., seven-stage) encoder structure 420; (2) a multi-stage (e.g., seven-stage) decoder structure 430; and (3) a feature map fusion module or block 440 coupled or attached to the decoder stages 430.

For the encoder stages 420, example configurations of the set of encoder blocks 420 are shown in Table 1 in FIG. 5. For the decoder stages 430, example configurations of the set of decoder blocks 430 are also shown in Table 1 in FIG. 5. As mentioned hereinbefore, “7”, “6”, “5” and “4” denote the heights (L) of the AC-RSU blocks. For example, the L may be configured according to the spatial resolution of the input feature maps. For feature maps with large height and width, greater L may be used to capture more large scale information. For example, the resolution of feature maps in En_6 and En_7 are relatively low, further downsampling of these feature maps leads to loss of useful context. Hence, in both En_6 and En_7 stages, AC-RSU-4F are used, where “F” denotes that the AC-RSU block is a dilated version, in which, for example, the pooling and upsampling operations are replaced with dilated convolutions. In this case, all of intermediate feature maps of AC-RSU-4F have the same resolution as its input feature maps.

For the decoder stages 430, example configurations of the set of decoder blocks (AC-RSU) are also shown in Table 1 in FIG. 5. In various example embodiments, the decoder stages 430 may have similar or corresponding structures to their symmetrical or corresponding encoder stages 420. For example, the dilated version AC-RSU-4F is also used for decoder blocks De_6 and De_7, which is similar or corresponding to that used for the symmetrical or corresponding encoder blocks En_6 and En_7. As shown in FIG. 4A, each decoder stage may be configured to take the concatenation of the upsampled feature map from its immediately previous stage and the downsampled feature map from its symmetrical or corresponding encoder stage as the inputs.

In various example embodiments, the prediction module 410 may be configured to generate a plurality of predicted feature maps based on the upsampled feature maps produced by the decoder stages 430. By way of example only and without limitation, in the example configuration shown in FIG. 4A, seven predicted feature maps (e.g., side output saliency probability maps) [symbol] _{side}, [symbol] _{side}, [symbol] _{side}, [symbol] _{side}, [symbol] _{side}, [symbol] _{side}, [symbol] _{side} from decoder stages De_1, De_2, De_3, De_4, De_5, De_6, De_7, respectively, may be produced based on a 3×3 convolution layer and a sigmoid function. Then, the prediction module 410 may upsample the logits (convolution outputs before sigmoid functions) of the side output saliency maps to the input image size and fuse them with a concatenation operation followed by a 1×1 convolution layer and a sigmoid function to generate the fused feature map (e.g., final saliency probability map) [symbol] _{fuse} 444.

Accordingly, the configuration of the ACU²-Net allows having deep architecture with rich multi-scale features and relatively low computation and memory costs. In addition, in various example embodiments, since the ACU²-Net architecture is built upon AC-RSU blocks without using any pre-trained backbones adapted from image classification, it is flexible and easy to be adapted to different working environments with insignificant performance loss.

Accordingly, in various example embodiments, the prediction module 410 has an encoder-decoder structure comprising a set of encoder blocks (e.g., En_1 to En_7) 420 and a set of decoder blocks (e.g., De_1 to De_7) 430. As shown in FIG. 4A, for each of a plurality of encoder blocks (e.g., En_1 to En_5) of the set of encoder blocks, a downsampled feature map may be produced using the encoder block based on an input feature map received by the encoder block. Furthermore, as shown in FIG. 4A, for each of a plurality of decoder blocks (e.g., De_1 to De_5) of the set of decoder blocks, an upsampled feature map may be produced using the decoder block based on an input feature map and the downsampled feature map produced by the encoder block corresponding to the decoder block received by the decoder block. Accordingly, in various example embodiments, a plurality of predicted feature maps produced based on the plurality of decoder blocks have different spatial resolution levels.

In various example embodiments, the plurality of predicted feature maps are produced based on the plurality of upsampled feature maps produced by the plurality of decoder blocks, respectively.

### Attentive Coordinate Convolution (AC-Conv)

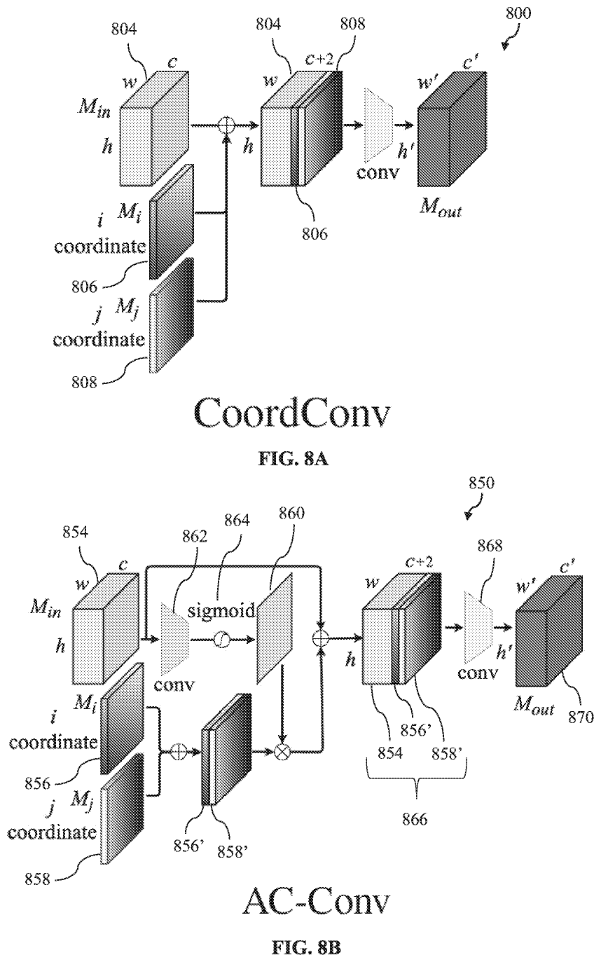

Various example embodiments note that soft-tissue structures like thyroids in medical images appear to have predictable position and shape patterns, which can be used to assist the segmentation process. There has been disclosed coordinate convolution (CoordConv) as shown in FIG. 8A to solve the coordinate transform problem (see Liu et al., “An intriguing failing of convolution neural networks and the CoordConv solution”, In NIPS, 9605-9616, 2018, herein referred to as the Liu reference, the content of which being hereby incorporated by reference in its entirety for all purposes). In particular, FIG. 8A depicts a schematic block diagram of the original CoordConv layer 800. Given an input feature map Mᵢₙ(h×ω×c) 804, CoordConv can be described as Mₒᵤₜ=conv(cat(Mᵢₙ, Mᵢ, Mⱼ)), where Mᵢ 806 and Mⱼ 808 denote the row and column coordinate maps, respectively. However, various example embodiments of the present invention note that since coordinate maps (Mᵢ, Mⱼ) attached to the features in different layers are almost constant, direct concatenation of them with feature maps Mᵢₙ in different layers may degrade the generalization capability of the network. This is because their corresponding convolution weights are responsible to synchronize their value scales with that of the feature map Mᵢₙ as well as extracting the geometric information. To address this issue, various example embodiments provide an attentive coordinate convolution (AC-Conv) 850 as shown in FIG. 8B. In particular, FIG. 8B depicts a schematic block diagram of the AC-Conv 850 according to various example embodiments of the present invention. The AC-Conv 850 adds a spatial-attention-like operation before the concatenation (channel-wise) of the input feature map 854 and the coordinate maps 856′, 858′ (corresponding to the plurality of weighted coordinate maps as described hereinbefore according to various embodiments):

$${M}_{out} = conv ( cat ( {M}_{in} , σ ( conv ( {M}_{in} ) ) \cdot cat ( {M}_{i} , {M}_{j} ) ) \qquad ( Equation 1 )$$

where σ is the sigmoid function.

Accordingly, in various example embodiments, performing a feature extraction operation using the convolution (AC-Conv) layer 850 comprises: producing the output feature map 870 of the convolution layer 850 based on an input feature map 854 received by the convolution layer 850 and a plurality of weighted coordinate maps 856′, 858′; producing the plurality of weighted coordinate maps 856′, 858′ based on a plurality of coordinate maps 856, 858 and a spatial attention map 860; and producing the spatial attention map 860 based on the input feature map 854 received by the convolution layer 850 for modifying coordinate information in each of the plurality of coordinate maps 856, 858 to produce the plurality of weighted coordinate maps 856′, 858′. In various example embodiments, producing the spatial attention map 860 comprises performing a first convolution operation 862 based on the input feature map 854 received by the convolution layer 850 to produce a convolved feature map; and applying an activation function 864 based on the convolved feature map to produce the spatial attention map 860. In various example embodiments, producing the plurality of weighted coordinate maps 856′, 858′ comprises multiplying each of the plurality of coordinate maps 856, 858 with the spatial attention map 860 so as to modify the coordinate information in each of the plurality of coordinate maps 856, 858. In various example embodiments, producing the output feature map 870 of the convolution layer 850 comprises: concatenating the input feature map 854 received by the convolution layer 850 and the plurality of weighted coordinate maps 856′, 858′ channel-wise to form a concatenated feature map 866; and performing a second convolution operation 868 based on the concatenated feature map 866 to produce the output feature map 870 of the convolution layer 850.

The spatial-attention-like operation plays two roles: i) as a synchronizing layer to reduce the scale difference between Mᵢₙ and {Mᵢ, Mⱼ}; ii) re-weights every pixel's coordinates, rather than using the constant coordinate maps, to capture more important geometric information with the guidance of the attention map 860 derived from the current input feature map 854. For example, for two coordinates, i and j, an i coordinate map (or i coordinate channel) 856 and a j coordinate map (or j coordinate channel) 858 may be provided. For example, i coordinate map 856 may be an h×ω rank-1 matrix with its first row filled with zeros (0s), its second row filled with ones (1s), its third row filled with twos (2s), and so on. The j coordinate map 858 may be the same or similar as i coordinate map 856 but with columns filled in with the above-mentioned values instead of rows. As described hereinbefore according to various example embodiments, the RSU 720 used in the U²-Net may be modified or adapted by replacing their convolution layers with the AC-Conv layer 850 according to various example embodiments to produce or build the AC-RSU according to various example embodiments. Compared with the RSU 720, for example, the AC-RSU is able to extract both texture and geometric features from different receptive fields. In various example embodiments, the prediction module ACU²-Net 410 and three sub-networks ACU²-Net-Ref7, ACU²-Net-Ref5 and ACU²-Net-Ref3 in the refinement E-module 450 are all built upon the AC-RSU.

### Parallel Multi-Head Residual Refinement Module (MH-RRM)

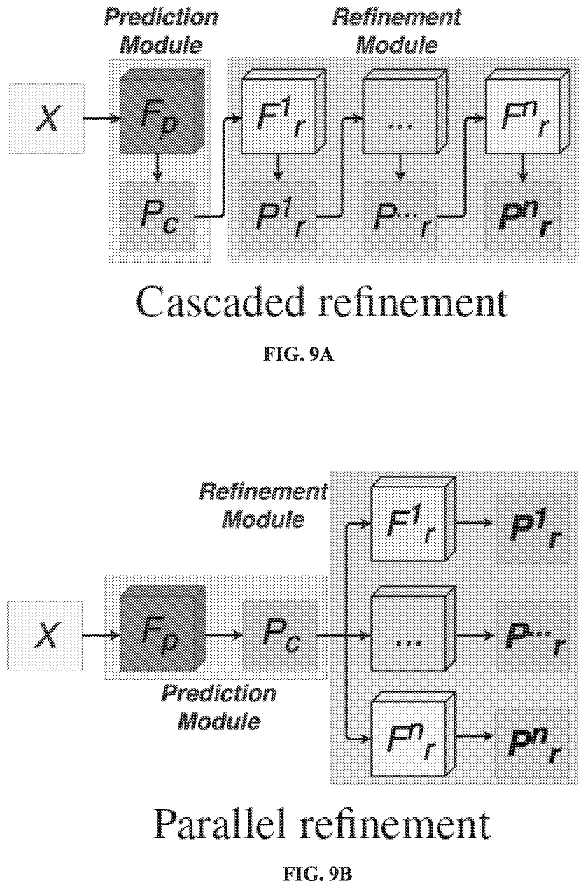

In an attempt to further improve the accuracy, a number of conventional predict-refine models have been designed to recursively or progressively refine the coarse result by cascaded sub-networks (cascaded refinement module): P_{c}=Fₚ(X), Pᵣ¹=Fᵣ¹(P_{c}), . . . , pᵣⁿ=Fᵣⁿ(Pᵣⁿ⁻¹), as shown in FIG. 9A. The last output Pᵣⁿ is, theoretically, the most accurate one and hence is usually taken as the final result. This cascaded refinement strategy is able to reduce the biases of segmentation results. However, various example embodiments found that the segmentation of soft-tissues in ultrasound images using such networks in practice often have large variances due to low image quality and blurry boundaries. Multi-model ensemble strategy can be used to reduce the prediction biases and variances. However, various example embodiments found that direct ensembling of multiple deep models requires heavy computation and time costs. To address these problems associated with conventional techniques, various example embodiments embed the ensemble strategy into the refinement module. In particular, a simple and effective parallel multi-head residual refinement module (MH-RRM) 450 as shown in FIG. 4B is provided according to various example embodiments of the present invention. By way of example only and without limitation, the number of the MH-RRM heads 454-1, 454-2, 454-3 (e.g., corresponding to the plurality of refinement blocks as described hereinbefore according to various embodiments) according to various example embodiments is set to three {Fᵣ¹, Fᵣ², Fᵣ³}, as shown in FIG. 4B. As described hereinbefore, the three refinement heads or blocks 454-1, 454-2, 454-3 may each be formed based on an ACU²-Net configured to produce a refined feature map having a different spatial resolution level based on the fused feature map 444. In various example embodiments, the plurality of refinement blocks 454-1, 454-2, 454-3 produce a plurality of refined feature maps 464-1, 464-2, 464-3, respectively. Accordingly, in various example embodiments, the plurality of refined feature maps 464-1, 464-2, 464-3 have different spatial resolution levels.

In various example embodiments, each of the plurality of refinement blocks 454-1, 454-2, 454-3 has an encoder-decoder structure comprising a plurality of encoder blocks and a plurality of decoder blocks. For each refinement block, and for each of the plurality of encoder blocks of the refinement block, as shown in FIG. 4B, a downsampled feature map may be produced using the encoder block based on an input feature map received by the encoder block. Furthermore, for each refinement block and for each of the plurality of decoder blocks of the refinement block, as shown in FIG. 4B, an upsampled feature map may be produced using the decoder block based on an input feature map and the downsampled feature map produced by the encoder block corresponding to the decoder block received by the decoder block. In various example embodiments, the plurality of encoder-decoder structures of the plurality of refinement blocks have different heights.

In various example embodiments, as shown in FIG. 4B, for each refinement block, the refined feature map of the refinement block may be produced based on the fused feature map 444 received by the refinement block and the upsampled feature map produced by a first decoder block 458-1, 458-2, 458-3 of the plurality of decoder blocks of the refinement block. In various example embodiments, the output image of the example CNN 400 is produced based on an average of the set of refined feature maps 464-1, 464-2, 464-3.

Accordingly, in various example embodiments, given an input image X, the final segmentation result of the example CNN 400 can be expressed as:

$${P}_{c} = {F}_{p} ( X ) \\ {P}_{r}^{1} = {F}_{r}^{1} ( {P}_{c} ) , {P}_{r}^{2} = {F}_{r}^{2} ( {P}_{c} ) , {P}_{r}^{3} = {F}_{r}^{3} ( {P}_{c} ) \\ {P}^{r} = avg ( {P}_{r}^{1} , {P}_{r}^{2} , {P}_{r}^{3} ) \qquad ( Equation 2 )$$

FIG. 9B illustrates a semantic workflow of the predict-refine architecture of the example CNN 400 with the above-mentioned parallel refinement module. In FIGS. 9A and 9B, the bold fonts indicate the final prediction results.

### Training and Inference

In the training process, the three refinement outputs, R⁽¹⁾ 464-1, R⁽²⁾ 464-2 and R⁽³⁾ 464-3, of the E-module 450 are supervised with independently computed losses, along with the seven side outputs S⁽ⁱ⁾ (i={1, 2, 3, 4, 5, 6, 7}) and one fused output S^{fuse} 444 from the prediction module 410, as shown in FIGS. 4A and 4B. The whole model may be trained end-to-end with Binary Cross Entropy (BCE) loss:

$$ℒ = {\sum }_{i = 1}^{I} {λ}^{{S}^{( i )}} {ℒ}^{{S}^{( i )}} + {λ}^{{S}^{( fuse )}} {ℒ}^{{S}^{( fuse )}} + {\sum }_{j = 1}^{J} {λ}^{{R}^{( j )}} {{ℒ}^{R}}^{( j )} \qquad ( Equation 3 )$$

where [symbol] is the total loss, [symbol] , [symbol] and [symbol] are the corresponding losses of the side outputs, fused output and refinement outputs. λ^{S}^{⁽ⁱ⁾}, λ^{S}^{^{(fuse)}} and λ^{R}^{^{(j)}} are their corresponding weights to emphasize different outputs. In experiments conducted according to various example embodiments, all the A weights are set to 1.0. In the inference process, the average of R⁽¹⁾ 464-1, R⁽²⁾ 464-2 and R⁽³⁾ 464-3 is taken as the final prediction result (e.g., corresponding to the output image of the CNN as described hereinbefore according to various embodiments).

### Experiments

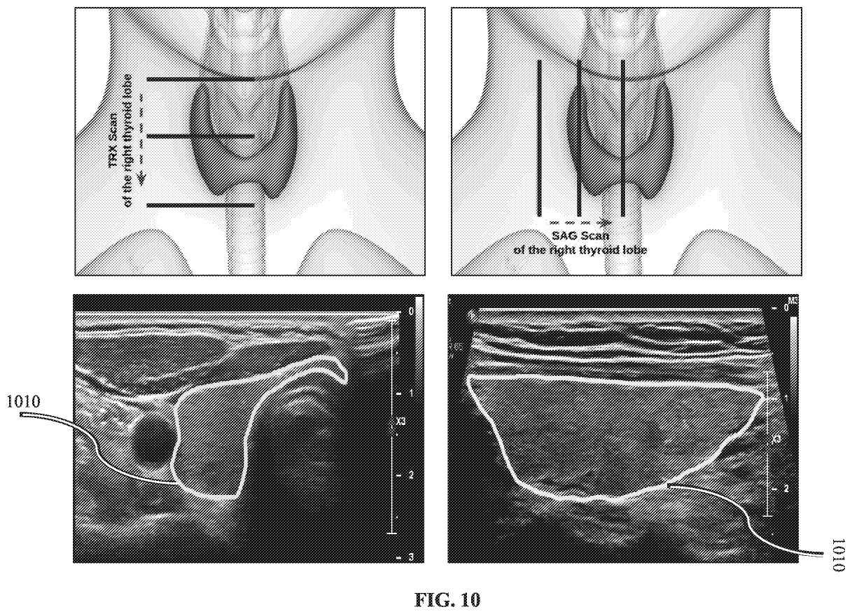

The thyroid gland is a butterfly-shaped organ at the base of the neck just superior to the clavicles, with left and right lobes connected by a narrow band of tissue in the middle called isthmus (see FIG. 10). In particular, FIG. 10 depicts a schematic drawing of the thyroid gland and ultrasound scanning protocol, along corresponding ultrasound images with manually labelled thyroid lobe overlay 1010. The dotted arrows in top row of images in FIG. 10 denote the scanning direction of ultrasound probe in the transverse (TRX) and sagittal (SAG) planes. The bottom row of images in FIG. 10 shows sample TRX (left) and SAG (right) images with manually labelled thyroid lobe overlay 1010.

To diagnose thyroid abnormalities, clinicians may asses its size by segmenting the thyroid gland manually from collected ultrasound scans. By way of example only for illustration purpose and without limitation, the example CNN 400 was evaluated on thyroid tissue segmentation problem as a case study.

### Dataset

It appears that none of the existing public datasets are suitable for large-scale learning based methods. To enable large-scale clinical applications, a comprehensive thyroid ultrasound segmentation dataset was collected with the approval of the health research ethics boards of the participating centers.

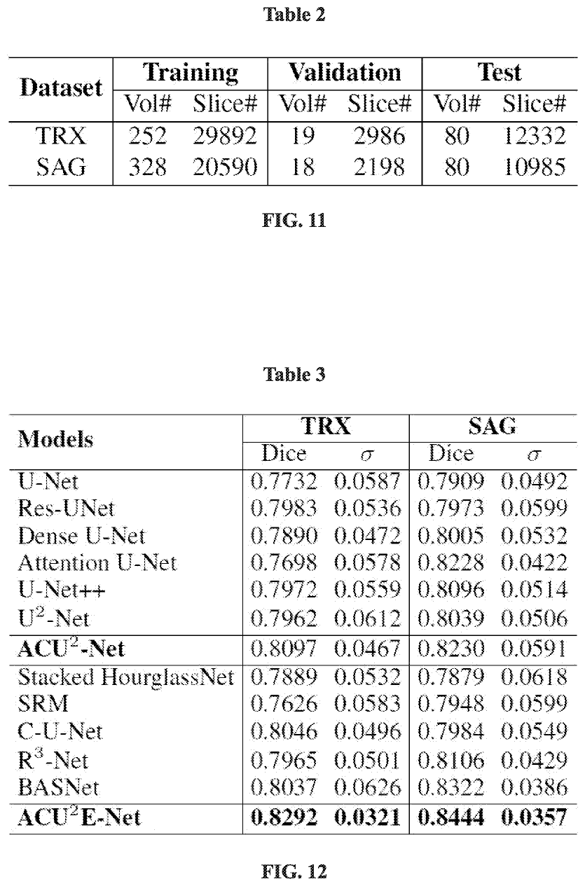

In relation to ultrasound scans collection, 777 ultrasound scans were retrospectively collected from 700 patients aged between 18 to 82 years who presented at 12 different imaging centers for a thyroid ultrasound examination. Scans were divided by the scanning direction of ultrasound probe in the transverse (TRX) and sagittal (SAG) planes (e.g., see FIG. 10). Hence two divisions (TRX and SAG sets) were available. Each division was randomly split further into three subsets for training, validating and testing, based on patient ID, so no same patient will end up in two different subsets. FIG. 11 depicts a table (Table 2) illustrating the number of volumes and the corresponding slices (images) in each subset. In particular, Table 2 shows the number of TRX and SAG thyroid scans in the thyroid datasets, whereby “Vol #” and “Slice #” denote the number of volumes and the corresponding labeled images, respectively.

In relation to annotation or labelling, images in the dataset were manually labeled by five experienced sonographers and verified by three radiologists. Given the rather large number of images available in total, ultrasound scans in the training sets were labeled every three or five slices to save labeling time. However, the validation and test sets were labeled slice-by-slice for accurate volumetric evaluation.

In relation to implementation details, the example CNN 400 was implemented with PyTorch. The designated train, valid and test set was used to evaluate the performance of the example CNN 400. In the training process, the input images were firstly resized to 160×160×3 and are then randomly cropped to 144×144×3. Online random horizontal and vertical flipping were used to augment the dataset. The training batch size was set to 12. The model weights were initialized by the default He uniform initialization (e.g., see He et al., “Delving deep into rectifiers: Surpassing human-level performance on imagenet classification”, In Proceedings of the IEEE international conference on computer vision, 1026-1034, 2015). Adam optimizer (e.g., see Kingma, “Adam: A method for stochastic optimization”, arXiv preprint arXiv:1412.6980, 2014) was used with a learning rate of 1e-3 and no weight decay. The training loss converges after around 50,000 iterations, which took about 24 hours. In the testing process, input images were resized to 160×160×3 and fed into the example CNN. Bilinear interpolation was used in both down-sampling and up-sampling process. Both the training and testing process were conducted on a 12-core, 24-thread PC with an AMD Ryzen Threadripper 2920×4.3 GHZ CPU (128 GB RAM) with an NVIDIA GTX 1080 Ti GPU.

In relation to evaluation metrics, two measures were used to evaluate the overall performance of the present method: Volumetric Dice (e.g., see Popovic et al., “Statistical validation metric for accuracy assessment in medical image segmentation”, IJCARS, 2(2-4): 169-181, 2007) and its standard deviation σ. The Dice score is defined as:

$$Dice = \frac{2 * \vert P ⋂ G \vert}{\vert P \vert + \vert G \vert} \qquad ( Equation 4 )$$

where P and G indicate the predicted segmentation mask sweep (h×ω×c) and the ground truth mask sweep (h×ω×c), respectively. The standard deviation of the Dice scores is computed as:

$$σ = \sqrt{\frac{1}{N} {\sum }_{i = 1}^{N} {( {Dice}_{i} - {Dice}_{μ} )}^{2}} \qquad ( Equation 5 )$$

where N is the number of testing volumes and Dice_{μ} denotes the average volumetric Dice score of the whole testing set. In the experiments conducted, the mean dice (Dice) of each testing set was reported along with the standard deviation (o).

The example CNN (ACU²E-Net) 400 was compared with 11 state-of-the-art (SOTA) models including U-Net (Ronneberger et al., “U-net: Convolutional networks for biomedical image segmentation”, In MICCAI, 234-241, 2015) and its five variants, including Res U-Net (e.g., see Xiao et al., “Weighted Res-UNet for high-quality retina vessel segmentation”, In ITME, 327-331, 2018), Dense U-Net (e.g., see Guan et al., “Fully Dense UNet for 2-D Sparse Photoacoustic Tomography Artifact Removal”, IEEE JBHI, 24(2): 568-576, 2019), Attention U-Net (e.g., see Oktay et al., “Attention u-net: Learning where to look for the pancreas”, arXiv preprint arXiv: 1804:03999, 2018), U-Net++ (e.g., see Zhou et al., “Unet++: A nested u-net architecture for medical image segmentation”, In MICCAI-W, 3-11, 2018) and U²-Net (e.g., see Qin et al., “U²-Net: Going Deeper with nested U-structure for salient object detection, Pattern Recognition, 106: 107404, 2020), as well as five predict-refine models including Stacked HourglassNet (e.g., see Newell et al., “Stacked hourglass networks for human pose estimation, In ECCV, 483-499”, 2016), SRM (e.g., see Wang et al., “A stagewise refinement model for detecting salient objects in images”, In ICCV, 4019-4028, 2017), C-U-Net (e.g., see Tang et al., “Quantized densely connected u-nets for efficient landmark localization”, In ECCV, 339-354, 2018), R³-Net (Deng et al., “R3net: Recurrent residual refinement network for saliency detection, In AAAI, 2018) and BASNet (Qin et al., “Basnet: Boundary-aware salient object detection”, In CVPR, 7479-7489, 2019).

FIG. 12 depicts a table (Table 3) showing the quantitative evaluation or comparison of the example CNN 400 with other state-of-the-art segmentation models on TRX and SAG test sets. The top part of Table 3 includes the comparisons against the classical U-Net and its variants like Attention U-Net, while the bottom part of the table shows the comparisons against the models involving predict-refine strategy like R³-Net. It can be observed that the example CNN 400 produces the highest DICE score on both TRX and SAG images. Furthermore, the parallel refinement module 450 greatly improves the Dice score by 2.55%, 1.22% and reduces the standard deviation σ by 31.99%, 7.51% against the second best model (BASNet) and other refinement module designs like R³-Net.

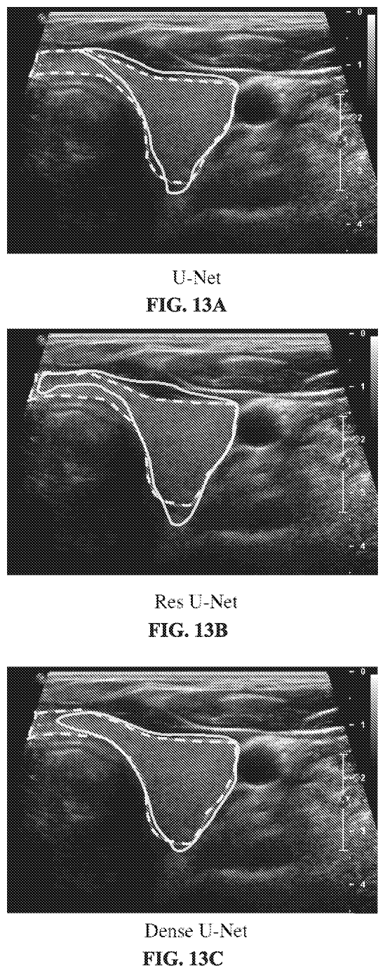

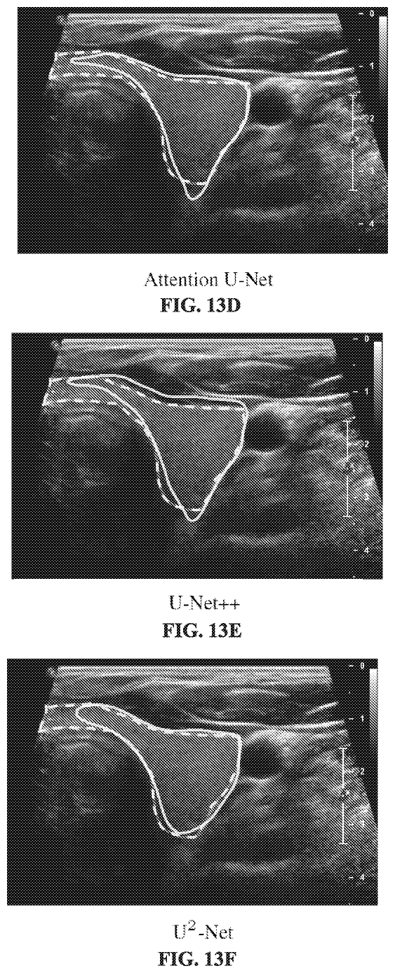

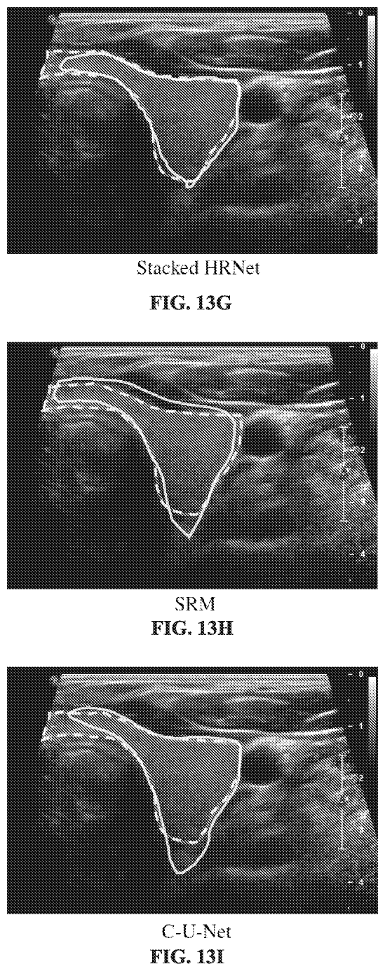

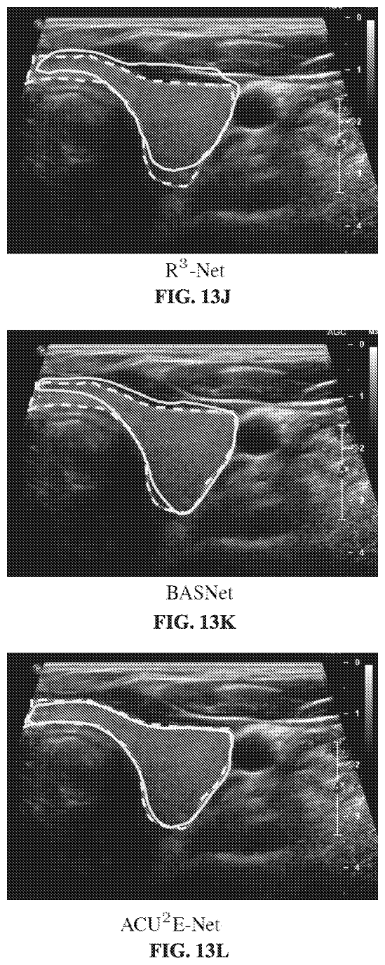

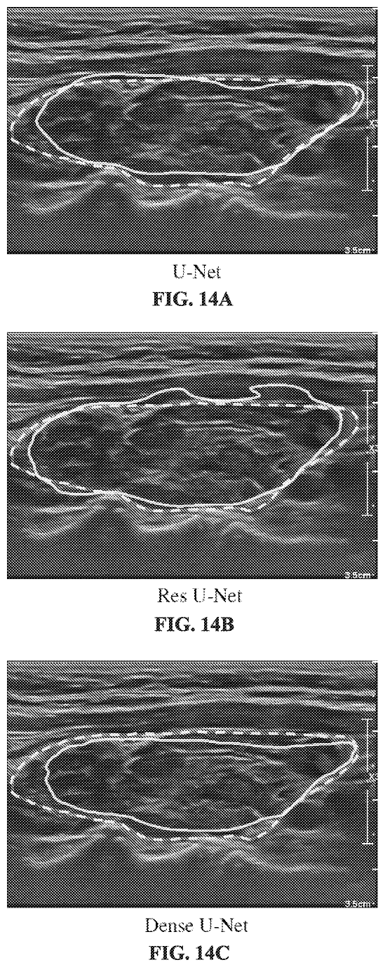

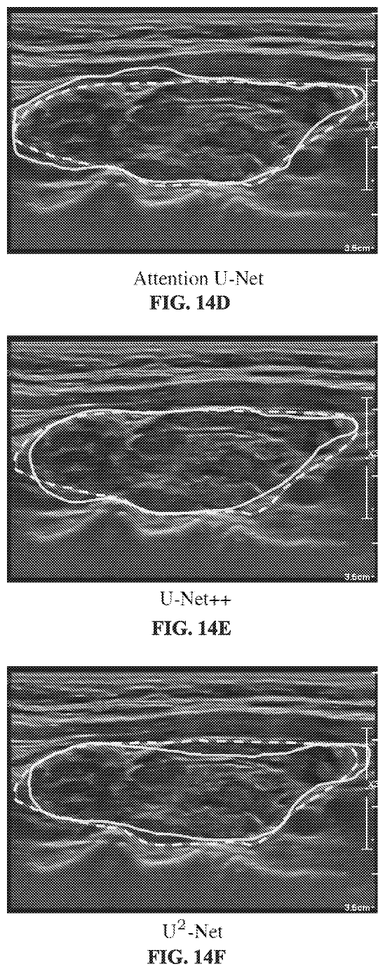

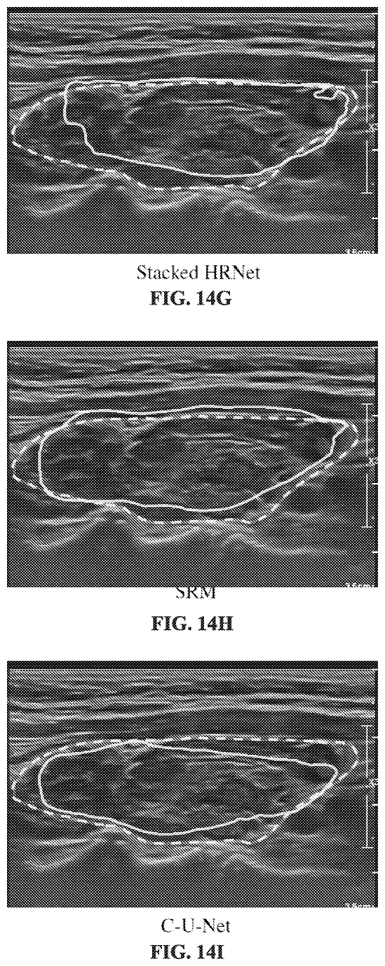

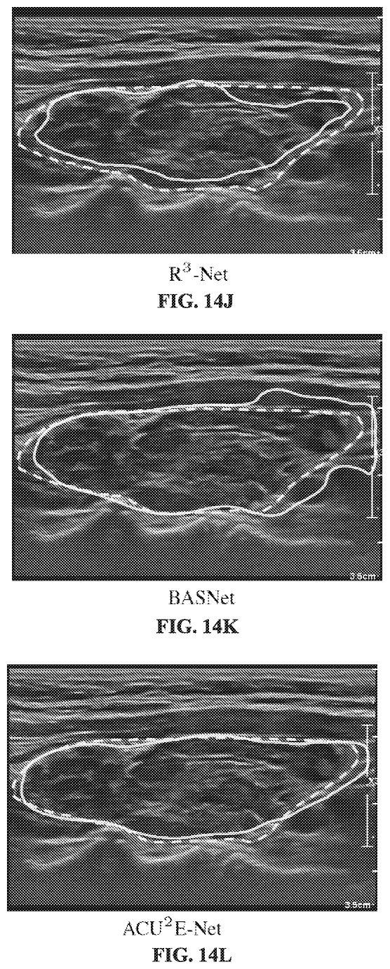

FIGS. 13A to 13L and 14A to 14L illustrate the sample segmentation results on TRX and SAG thyroid images. In particular, FIGS. 13A to 13L depict a qualitative comparison of ground truth (dotted white line) and segmentation results (full white line) for different methods on a sampled TRX slice with homogeneous thyroid, and FIG. 14A to 14L depict a qualitative comparison of ground truth (dotted white line) and segmentation results (full white line) for different methods on a sampled SAG slice with heterogeneous thyroid. As can be seen, the example CNN 400 was able to produce improved (more accurate) segmentation results. Specifically, FIGS. 13A to 13L show a homogeneous TRX thyroid lobe with heavy sparkle noises and blurry boundaries. Res U-Net, U-Net++, SRM, C-U-Net, R³-Net and BASNet fail to capture the accurate boundary. Other models, such as U-Net, Dense U-Net, Attention U-Net, U²-Net and Stacked HourglassNet fail in segmenting the top-left elongated region of the thyroid. FIGS. 14A to 14L illustrates the segmentation results of a heterogeneous SAG view thyroid, which contains several complicated nodules. Accordingly, as can be seen, the example CNN 400 produces relatively better results than other models.

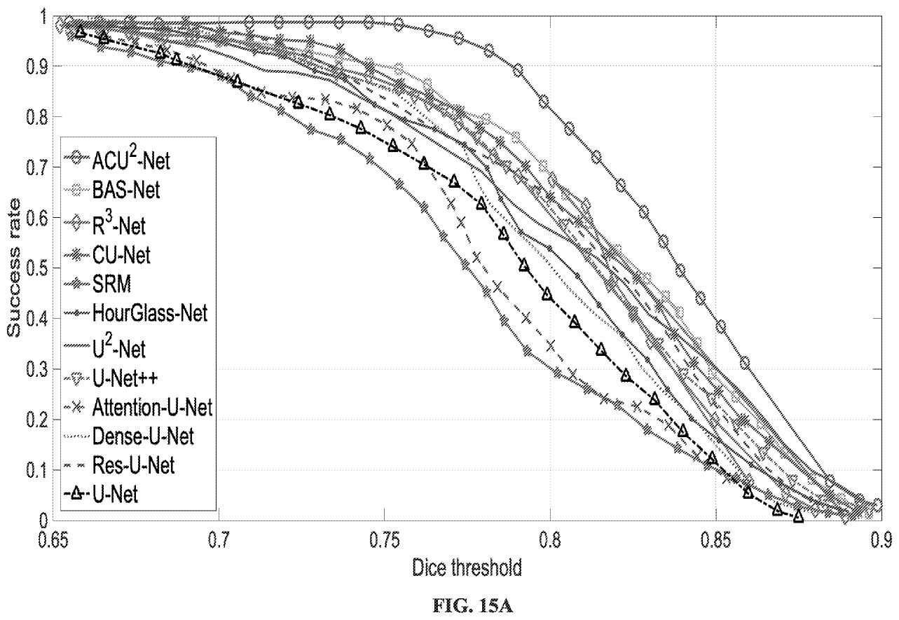

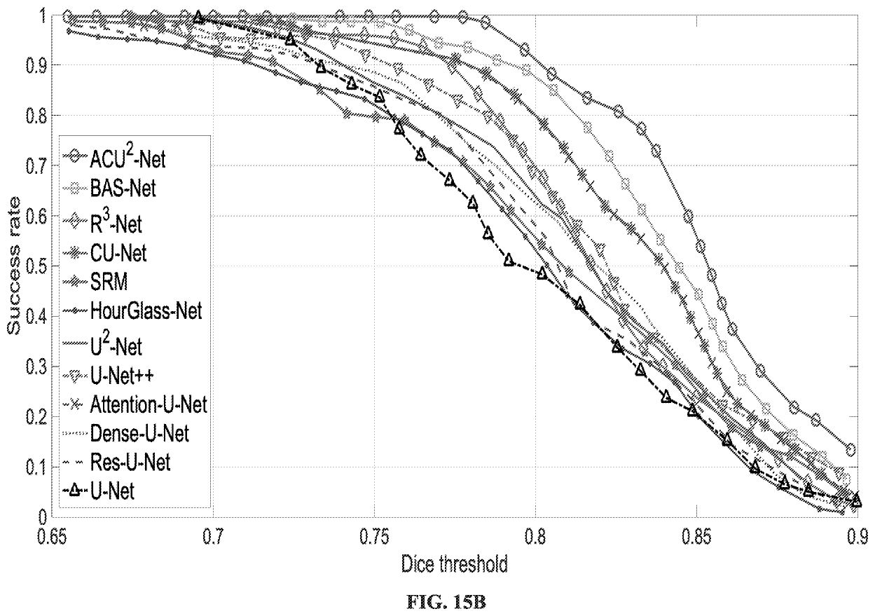

To further evaluate the robustness of the example CNN 400, the success rate curves of the example CNN 400 and the other 11 state-of-the-art models on TRX images and SAG images are plotted in FIGS. 15A and 15B, respectively. The success rate is defined as the ratio of number of scan predictions (with scores higher than certain dice thresholds) over the total number of scans. Higher success rate denotes better performance and hence the top curve (ACU²E-Net) is better than the others 11 state-of-the-art models being compared. Accordingly, as can be seen, the example CNN 400 outperforms other models on both TRX and SAG test sets by large margins.

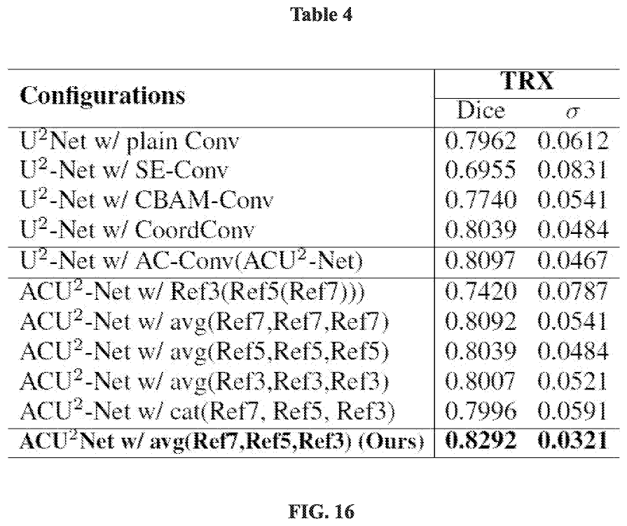

To validate the effectiveness of the AC-Conv according to various example embodiments, the ablation studies were conducted by replacing the plain convolution (plain Conv) (LeCun et al., “Gradient-based learning applied to document recognition”, Proceedings of IEEE, 86(11): 2278-2324, 1998) in adapted U²-Net with the following variants: SE-Conv (Hu et la., “Squeeze-and-excitation networks”, In CVPR, 7132-7141, 2018) which explicitly model channel inter-dependencies by its squeeze-and-excitation block, CBAM-Conv (Woo et al., “Cbam: Convolutional block attention module”, In ECCV, 3-19, 2018) which refines feature maps with its channel and spatial attention blocks, CoordConv (Liu et al., “An intriguing failing of convolution neural networks and the CoordConv solution”, In NIPS, 9605-9616, 2018) which gives convolution access to its own input coordinates through the use of coordinate channels, and our AC-Conv. FIG. 16 depicts a table (Table 4) showing the ablation studies conducted on different convolution blocks and refinement architectures. In Table 4, Ref7 is the abbreviation of ACU²-Net-Ref7. The experiments were conducted on TRX thyroid test set. The results on TRX test set are shown in the top part of Table 4. As can be seen, the ACU²-Net using AC-Conv gives the best results in terms of both Dice score and standard deviation σ. This further demonstrates that a combined strategy of jointly perceiving geometric and spatial information is more effective than standalone spatial attention-based (CBAM) or coordinate-based (CoordConv) methods.

To validate the performance of the MH-RRM (E-module), the ablation studies were also conducted on different refinement configurations including cascaded RRM Ref3(Ref5 (Ref7))), parallel RRM with three same RRM avg(Refk, Refk, Refk) {k=3, 5, 7} and fused parallel RRM conv(Ref7, Ref5, Ref3), where the parallel refinement outputs are fused by a convolution layer instead of averaging in the inference. The bottom part of Table 4 shows the ablation results on RRM, which indicate that the cascaded RRM, the parallel RRM with the same branches as well as the fused parallel RRM are all inferior to the MH-RRM according to various example embodiments.

Accordingly, various example embodiments advantageously provide an attention-based predict-refine network (ACU²E-Net) 400 for segmentation of soft tissues structures in ultrasound images. In particular, the ACU²E-Net is built upon (a) the attentive coordinate convolution (AC-Conv) 850, which makes full use of the geometric information of the thyroid gland in ultrasound images, and (b) the parallel multi-head refinement module (MH-RRM) 450 which refines the segmentation results by integrating the ensemble strategy with a residual refinement approach.

The thorough ablation studies and comparisons with state-of-the-art models described hereinbefore demonstrate the effectiveness and robustness of the example CNN 400, without complicating the training and inference processes. Although the example CNN 400 has been described with respect to segmentation of thyroid tissue from ultrasound images, it will be appreciated that the example CNN 400, as well as the AC-Conv 850 and MH-RRM 450, is not limited to being applied to segment thyroid tissue from ultrasound images, and can be applied to segment other types of tissues from ultrasound images as desired or as appropriate, such as but not limited to liver, spleen, and kidneys, as well as tumors (e.g., Hepatocellular carcinoma (HCC) in the liver or subcutaneous masses).

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. A method of image processing based on a convolutional neural network (CNN), using at least one processor, the method comprising:
receiving an input image;
performing a plurality of feature extraction operations using a plurality of convolution layers of the CNN to produce a plurality of output feature maps, wherein a respective feature extraction operation of the plurality of feature extraction operations is performed by a respective convolution layer of the plurality of convolution layers and includes:
receiving, by the respective convolution layer, a respective input feature map and a plurality of coordinate maps;
generating, by the respective convolution layer, a respective spatial attention map based on the respective input feature map;
generating, by the respective convolution layer, a plurality of weighted coordinate maps based on the plurality of coordinate maps and the respective spatial attention map; and
outputting, by the respective convolution layer, a respective output feature map of the respective convolution layer based on the respective input feature map and the plurality of weighted coordinate maps; and
producing an output image corresponding to the input image based on the plurality of output feature maps of the plurality of convolution layers.

2. The method according to claim 1, wherein generating, by the respective convolution layer, the respective spatial attention map based on the respective input feature map comprises:
performing a first convolution operation based on the respective input feature map received by the respective convolution layer to produce a respective convolved feature map; and
applying an activation function based on the respective convolved feature map to generate the respective spatial attention map.

3. The method according to claim 2, wherein the activation function is a sigmoid activation function.

4. The method according to claim 2, wherein generating, by the respective convolution layer, the plurality of weighted coordinate maps comprises multiplying each of the plurality of coordinate maps with the respective spatial attention map so as to modify coordinate information in each of the plurality of coordinate maps.

5. The method according to claim 2, wherein the plurality of coordinate maps comprises a first coordinate map comprising coordinate information with respect to a first dimension and a second coordinate map comprising coordinate information with respect to a second dimension, the first and second dimensions being two dimensions over which the first convolution operation is configured to perform.

6. The method according to claim 1, wherein outputting, by the respective convolution layer, the respective output feature map of the respective convolution layer comprises:
concatenating the respective input feature map received by the respective convolution layer and the plurality of weighted coordinate maps channel-wise to form a respective concatenated feature map; and
performing a second convolution operation based on the respective concatenated feature map to produce the respective output feature map of the respective convolution layer.

7. The method according to claim 1, wherein:
the CNN comprises a prediction sub-network comprising at least one convolution layer of the plurality of convolution layers of the CNN; and
the method further comprises:
producing a set of predicted feature maps using the prediction sub-network based on the input image, including:
performing at least one feature extraction operation, of the plurality of feature extraction operations, using the at least one convolution layer of the prediction sub-network, wherein the set of predicted feature maps include a plurality of predicted feature maps having different spatial resolution levels.

8. The method according to claim 7, wherein:
the prediction sub-network has an encoder-decoder structure comprising a plurality of first encoder blocks and a plurality of first decoder blocks, each first encoder block of the plurality of first encoder blocks corresponding to one respective first decoder block of the plurality of first decoder blocks, and
the method further comprises:
producing, by a respective first encoder block of the plurality of first encoder blocks, a respective downsampled feature map based on a respective input feature map received by the respective first encoder block; and
producing, by a respective first decoder block, of the plurality of first decoder blocks, corresponding to the respective first encoder block, a respective upsampled feature map based on the respective input feature map and the respective downsampled feature map produced by the respective first encoder block corresponding to the respective first decoder block.

9. The method according to claim 8, wherein producing the set of predicted feature maps using the prediction sub-network comprises producing the plurality of predicted feature maps based on a plurality of upsampled feature maps produced by the plurality of first decoder blocks.

10. The method according to claim 8, wherein:
for a respective first encoder block of the plurality of first encoder blocks, producing the respective downsampled feature map comprises:
extracting first multi-scale features based on the respective input feature map received by the respective first encoder block; and
producing the respective downsampled feature map based on the extracted first multi-scale features, and
for a respective first decoder block of the plurality of first decoder blocks, producing the respective upsampled feature map comprises:
extracting second multi-scale features based on the respective input feature map and the respective downsampled feature map produced by the respective first encoder block corresponding to the respective first decoder block received by the decoder block; and
producing the respective upsampled feature map based on the extracted second multi-scale features extracted by the respective decoder block.

11. The method according to claim 8, wherein:
each of the plurality of first encoder blocks of the prediction sub-network comprises at least one convolution layer of the plurality of convolution layers of the CNN; and
producing, by the respective first encoder block of the plurality of first encoder blocks, the respective downsampled feature map includes:
performing at least one feature extraction operation of the plurality of feature extraction operations using the at least one convolution layer of the respective first encoder block; and
each of the plurality of first decoder blocks of the prediction sub-network comprises at least one convolution layer of the plurality of convolution layers of the CNN; and
producing, by the respective first decoder block of the plurality of first decoder blocks, the respective upsampled feature map includes:
performing at least one feature extraction operation of the plurality of feature extraction operations using the at least one convolution layer of the respective first decoder block.

12. The method according to claim 11, wherein:
each convolution layer of each of the plurality of first encoder blocks of the prediction sub-network is one of the plurality of convolution layers of the CNN, and
each convolution layer of each of the plurality of first decoder blocks of the prediction sub-network is one of the plurality of convolution layers of the CNN.

13. The method according to claim 8, wherein:
each of the plurality of first encoder blocks of the prediction sub-network is configured as a residual block, and
each of the plurality of first decoder blocks of the prediction sub-network is configured as a residual block.

14. The method according to claim 7, wherein:
the CNN further comprises a refinement sub-network comprising at least one convolution layer of the plurality of convolution layers of the CNN,
the method further comprises producing a set of refined feature maps using the refinement sub-network based on a fused feature map, the producing including:
performing at least one feature extraction operation of the plurality of feature extraction operations using the at least one convolution layer of refinement sub-network, wherein the set of refined feature maps includes a plurality of refined feature maps having different spatial resolution levels.

15. The method according to claim 14, further comprising concatenating the set of predicted feature maps to produce the fused feature map.

16. The method according to claim 14, wherein the refinement sub-network comprises a plurality of refinement blocks configured to produce the plurality of refined feature maps, each of the plurality of refinement blocks having an encoder-decoder structure comprising a plurality of second encoder blocks a plurality of second decoder blocks, wherein a respective second encoder block in the plurality of second encoder blocks corresponds to one respective second decoder block in the plurality of second decoder blocks, and
the method further comprises, for each refinement block of the plurality of refinement blocks:
producing, by each second encoder block of the plurality of second encoder blocks, a respective downsampled feature map using the respective second encoder block based on an input feature map received by the respective second encoder block; and
producing, by each second decoder block of the plurality of second decoder blocks, a respective upsampled feature map using the respective second decoder block based on the respective input feature map and the respective downsampled feature map produced by the respective second encoder block corresponding to the respective second decoder block and received by the respective second decoder block.

17. The method according to claim 16, wherein the plurality of refinement blocks comprises a plurality of encoder-decoder structures having different heights.

18. The method according to claim 16, wherein the plurality of refinement blocks is configured to produce the plurality of refined feature maps by:
producing, for each refinement block of the plurality of refinement blocks, a respective refined feature map of the plurality of refined feature maps based on the fused feature map received by the respective refinement block and a respective upsampled feature map produced by a respective second decoder block, of the plurality of second decoder blocks, corresponding to the respective refinement block.

19. The method according to claim 16, wherein:
producing, for each second encoder block of the plurality of second encoder blocks, the respective downsampled feature map comprises:
extracting first multi-scale features based on the respective input feature map received by the respective second encoder block; and
producing the respective downsampled feature map based on the extracted first multi-scale features extracted by the respective second encoder block, and
producing, for each second decoder block of the plurality of second decoder blocks, the respective upsampled feature map comprises:
extracting second multi-scale features based on the respective input feature map and the respective downsampled feature map produced by the respective second encoder block corresponding to the respective second decoder block and received by the respective second decoder block; and
producing the respective upsampled feature map based on the extracted second multi-scale features extracted by the respective second decoder block.

20. The method according to claim 16, wherein, for a respective refinement block of the plurality of refinement blocks:
each of the plurality of second encoder blocks corresponding to the respective refinement block comprises at least one convolution layer of the plurality of convolution layers of the CNN; and
producing, by each second encoder block of the plurality of second encoder blocks, the respective downsampled feature map using the respective second encoder block of the respective refinement block comprises:
performing at least one feature extraction operation of the plurality of feature extraction operations using the at least one convolution layer of the respective second encoder block; and
each of the plurality of second decoder blocks corresponding to the respective refinement block comprises at least one convolution layer of the plurality of convolution layers of the CNN; and
producing, by each second decoder block of the plurality of second decoder blocks, the respective upsampled feature map using the respective second decoder block of the respective refinement block comprises:
performing at least one feature extraction operation of the plurality of feature extraction operations using the at least one convolution layer of the respective second decoder block.

21. The method according to claim 20, wherein:
each convolution layer of each of the plurality of second encoder blocks of the refinement block is one of the plurality of convolution layers of the CNN, and
each convolution layer of each of the plurality of second decoder blocks of the refinement block is one of the plurality of convolution layers of the CNN.

22. The method according to claim 16, wherein, for each of the plurality of refinement blocks:
each of the plurality of second encoder blocks of the refinement block is configured as a residual block, and
each of the plurality of second decoder blocks of the refinement block is configured as a residual block.

23. The method according to claim 14, wherein the output image is produced based on the set of refined feature maps.

24. The method according to claim 23, wherein the output image is produced based on an average of the set of refined feature maps.

25. The method according to claim 1, wherein:
receiving the input image comprises receiving a plurality of input images, each of the plurality of input images being a labeled image so as to train the CNN to obtain a trained CNN, and
the method further includes, for each of the plurality of input images:
performing the plurality of feature extraction operations using the plurality of convolution layers of the CNN to produce the plurality of output feature maps; and
producing the output image corresponding to the input image based on the plurality of output feature maps of the plurality of convolution layers.

26. The method according to claim 25, wherein the label image is a labeled ultrasound image including a tissue structure.

27. The method according to claim 1, wherein the output image is a result of an inference on the input image using the CNN.

28. The method according to claim 27, wherein the input image is an ultrasound image including a tissue structure.

29. A system for image processing based on a convolutional neural network (CNN), the system comprising:
a memory; and
at least one processor communicatively coupled to the memory and configured to perform a set of operations, comprising:
receiving an input image;
performing a plurality of feature extraction operations using a plurality of convolution layers of the CNN to produce a plurality of output feature maps, wherein a respective feature extraction operation of the plurality of feature extraction operations is performed by a respective convolution layer of the plurality of convolution layers and includes:
receiving, by the respective convolution layer, a respective input feature map and a plurality of coordinate maps;
generating, by the respective convolution layer, a respective spatial attention map based on the respective input feature map;
generating, by the respective convolution layer, a plurality of weighted coordinate maps based on the plurality of coordinate maps and the respective spatial attention map; and
outputting, by the respective convolution layer, a respective output feature map of the respective convolution layer based on the respective input feature map and the plurality of weighted coordinate maps; and
producing an output image corresponding to the input image based on the plurality of output feature maps of the plurality of convolution layers.

30. A computer program product, embodied in one or more non-transitory computer-readable storage media, comprising instructions executable by at least one processor to perform a set of operations using a convolutional neural network (CNN), the set of operations comprising:
receiving an input image;
performing a plurality of feature extraction operations using a plurality of convolution layers of the CNN to produce a plurality of output feature maps, wherein a respective feature extraction operation of the plurality of feature extraction operations is performed by a respective convolution layer of the plurality of convolution layers and includes:
receiving, by the respective convolution layer, a respective input feature map and a plurality of coordinate maps;
generating, by the respective convolution layer, a respective spatial attention map based on the respective input feature map;
generating, by the respective convolution layer, a plurality of weighted coordinate maps based on the plurality of coordinate maps and the respective spatial attention map; and
outputting, by the respective convolution layer, a respective output feature map of the respective convolution layer based on the respective input feature map and the plurality of weighted coordinate maps; and
producing an output image corresponding to the input image based on the plurality of output feature maps of the plurality of convolution layers.

31. The method according to claim 1, further comprising segmenting a tissue structure in an ultrasound image using the CNN (CNN), using at least one processor, the method comprising:
wherein:
the input image is the ultrasound image including the tissue structure; and
the output image has the tissue structure segmented and is a result of an inference on the input image using the CNN.

32. (canceled)

33. (canceled)

34. (canceled)
